(19) United States
(12) Patent Application Publication
(10) Pub. No.: US 2022/0111155 A1
(43) Pub. Date: Apr. 14, 2022

(54) DEVICE FOR PREVENTING NEEDLE REUSE AND INJURY, SYRINGE, AND METHOD

(71) Applicant: INTACT PUR-NEEDLE LLC, New Milford, CT (US)

(72) Inventors: Daniel Py, Larchmont, NY (US); Julian V. Chan, New Milford, CT (US)

(21) Appl. No.: 17/497,907

(22) Filed: Oct. 9, 2021

Related U.S. Application Data
(60) Provisional application No. 63/090,003, filed on Oct. 9, 2020.
(60) Provisional application No. 63/125,340, filed on Dec. 14, 2020.

(51) Int. Cl. A61M 5/32; A61M 5/00

(52) U.S. Cl. CPC A61M 5/3232; A61M 2005/3128; A61M 5/002; A61M 5/3221

(57) ABSTRACT

Device for preventing needle reuse and injury has a needle cap or enclosure, a body, and a needle. The needle cap and/or body is movable relative to the other from (i) a first position wherein the needle is received within and enclosed by the needle cap to prevent needle injury; (ii) to a second position wherein the needle is passed through the needle cap to penetrate tissue; and (iii) to a third position wherein the needle is within and enclosed by the needle cap, and the needle cap and body are locked to prevent needle reuse and injury. The needle cap conceals the needle from view before, during and after the patient injection. An intra-needle one-way valve allows liquid flow therethrough in a direction from an inlet toward an outlet of the needle, and prevents liquid flow in an opposite direction to prevent cross-contamination between the needle and a syringe.

### CROSS-REFERENCE TO RELATED APPLICATIONS

This application claimed benefit under 35 U.S.C. § 119(e) of U.S. Provisional Application No. 63/090,003, filed Oct. 9, 2020, and U.S. Provisional Application No. 63/125,340, filed Dec. 14, 2020, the contents of which are hereby incorporated by reference in their entirety as part of the present disclosure.

### FIELD OF THE INVENTION

The present invention relates to injection needle safety, and more particularly, to devices and methods that prevent needle reuse and injury, and to syringes that can be used with such devices, including multiple dose syringes.

### BACKGROUND INFORMATION

One drawback or disadvantage associated with prior art medical needles and syringes is that the needles can be reused. Needle reuse can lead to significant problems, including exposure of the healthcare worker or practitioner to blood or other body fluids and cross contamination from one patient to the next or from patient to healthcare worker. Cross contamination typically involves an unintentional transfer of bacteria, microorganisms, plasma, such as plasma with HIV, or hepatitis, from one substance, object or person to another and can lead to dangerous infections and other harmful effects, including death. Another drawback or disadvantage is that the needles are sufficiently exposed prior to, during or following use such that they give rise to inadvertent needle sticks or sharps injuries. Some of these injuries occur during needle use, a significant number of such injuries occur after needle use but before disposal of the needle, during or after disposal of the needle, and at other times. Sharps injuries can likewise lead to significant problems, including exposure of the healthcare worker or practitioner to blood or other body fluids and cross contamination. The injection safety policy released by the World Health Organization (“WHO”) in February 2015 recommended the exclusive use of auto-disable/reuse-prevention/sharps injury prevention (AD/RUP/SIP) syringes for most medical injections and recommended to all WHO Member States, particularly those with a problem of unsafe injection, to switch to their use by 2020.

Another drawback or disadvantage associated with prior art needles and syringes is that the needle is visibly exposed and can be frightening for children and other persons who fear them. Some persons suffer from trypanophobia or needle phobia, which is an extreme fear of medical procedures involving injections or hypodermic needles. Other persons can tolerate needles but nevertheless are frightened by their appearance, particularly prior to and/or during an injection, which can provoke agitation of the patient and lead to more painful injections.

Yet another drawback of prior art syringes is that injections are performed by manually depressing a plunger into a barrel of the syringe. The plunger typically includes an elastic seal between the distal end of the plunger and the inner wall of the barrel. Accordingly, the speed of the injection and the force applied to the plunger to inject the substance into a patient can vary significantly from one healthcare worker or practitioner to another. When injecting vaccines, such as during a pandemic, healthcare workers can become tired and subjected to stress, and their ability to confidently handle and manipulate a prior art syringe can be compromised. Typical syringes are intended for single injections. Accordingly, when used for single injections, particulates resulting from the friction forces between the elastic plunger tip and rigid syringe barrel are negligible. However, a syringe designed for multiple actuations cannot allow for such frictional forces over the injection of multiple doses by the same syringe because it could lead to particle formation above an acceptable threshold.

It is an object of the present invention, and/or of the embodiments thereof, to overcome one or more of the above-described drawbacks and/or disadvantages of the prior art.

### SUMMARY OF THE INVENTION

In accordance with a first aspect, the present invention is directed to a device for preventing needle reuse and injury, comprising a needle enclosure or cap, a body, and a metal needle mounted between the needle cap and body. The metal needle defines an inlet and an outlet, and the needle cap and/or the body is movable relative to the other from (i) a first position wherein the metal needle is received within and enclosed by the needle cap to prevent needle injury and, if desired, prevent the needle from being visible to the patient; (ii) to a second position wherein at least a portion of the metal needle is passed through the needle cap to allow the needle to penetrate tissue while nevertheless being concealed form patient view; and (iii) to a third position wherein the metal needle is received within and enclosed by the needle cap, and the needle cap and body are locked in the third position to prevent needle reuse and injury. Preferably, the needle is concealed from view in the first, second and third positions. In addition, the needle is preferably automatically retracted into the third position by a spring, for example, and the needle cap and body are locked in the third position by a locking member in the form of a cam. The device further includes a one-way valve in fluid communication with the outlet of the metal needle. The one-way valve allows liquid or other fluid flow therethrough in a direction from the inlet toward the outlet of the needle, and substantially prevents liquid or other fluid flow therethrough in a direction from the outlet toward the inlet of the needle, to thereby prevent any liquid or other fluid within the needle from flowing backwards after possible contact with patient fluid, to thereby prevent the patient from retro-contaminating the needle inlet, and consequently cross contaminating the source of fluid, such as by flowing through the needle inlet and outside the device.

Some embodiments of the present invention further comprise a spring mounted between the needle cap and body that biases the needle cap and/or the body in a direction from a proximal end toward a distal end of the device. In some such embodiments, the spring is formed integral with the needle cap. In some embodiments, the spring is substantially coil-shaped.

In some embodiments of the present invention, the body includes a carrier or syringe mount configured to releasably mount the device to a syringe or other type of carrier. In some such embodiments, the syringe mount includes an inlet port in fluid communication with the one-way valve to allow fluid to flow from a syringe connected thereto to the needle one-way valve. In some such embodiments, the syringe mount comprises a needle support including the needle mounted thereon and defining the inlet port therein. A syringe mounting flange is spaced relative to the needle support and defines a gap therebetween for receiving a distal end portion of a syringe and releasably mounting the device thereto. In some such embodiments, the syringe mounting flange is configured to releasably mount the device to a syringe including on a distal end portion thereof one or more raised surface areas or recessed surface areas. The syringe mounting flange includes one or more raised surface areas or recessed surface areas engageable with the raised or recessed surfaces areas of the syringe to releasably mount the device to the syringe.

In some embodiments of the present invention, the one-way valve includes an elastic valve member, a relatively rigid valve seat, and an axially-elongated, normally-closed valve seam therebetween. In some such embodiments, the elastic valve member defines an interference fit with the valve seat, a valve inlet is located at approximately a proximal end of the valve seam, and a valve outlet is located at approximately a distal end of the valve seam. In some such embodiments, the one-way valve defines a valve-opening pressure based on the degree of interference between the elastic valve member and valve seat. In some embodiments, the valve seat is defined by an annular surface, and the elastic valve member is received within the annular surface and forms the interference fit therebetween. In some such embodiments, the annular surface tapers inwardly in a direction from the inlet toward the outlet of the one-way valve. In some embodiments, the needle includes a hollow base, and the elastic valve member is received therein between the valve seat and a needle mount. In some embodiments, the elastic valve member is compressed into engagement with the valve seat and the needle mount. In some embodiments, the elastic valve member defines on a proximal end thereof a recessed surface defining an inlet port in fluid communication between the normally-closed, axially-elongated valve seam and a fluid passageway in the needle mount. In some such embodiments, the elastic valve member forms a fluid-tight seal against the needle mount.

In some embodiments of the present invention, the body includes on an exterior surface thereof at least one protuberance engageable with an opening in a waste or sharps container to remove the device from a syringe and dispose of same in the waste or sharps container.

In some embodiments of the present invention, the needle cap includes a locking member defining a proximal end, a distal end, a first side located between the proximal and distal ends, and a second side opposite the first side and located between the proximal and distal ends. The body includes a locking arm. The locking arm slidably contacts the first side of the locking member during movement from the first positon to the second position, and slidably contacts the second side of the locking member during movement from the second position to the third position. In some such embodiments, the locking arm is biased into contact with the locking member. In some such embodiments, the locking arm defines a distal end that slidably contacts the locking member, a recess is defined on the second side and/or at or near the proximal end of the locking member, and in the third position, the distal end of the locking arm is biased into the recess to thereby lock the needle cap and body in the third position. In some such embodiments, the distal end of the locking arm is flexed into engagement with the locking member, and in the third position, the distal end of the locking arm is snapped into engagement with the recess of the locking member to thereby lock the needle cap and body in the third position. In some embodiments, the first and second sides of the locking member are oriented at one or more acute angles relative to each other.

In some embodiments of the present invention, the needle cap and/or body defines an axially-extending recessed surface, and the other of the needle cap and/or body defines an axially-extending raised surface received within the recessed surface and substantially preventing rotation of the needle cap and/or body relative to the other. In some such embodiments, the needle cap and body define two axially-extending recessed surfaces and two axially-extending raised surfaces received within respective recessed surfaces.

In accordance with another aspect, the present invention is directed to any of the devices as described above in combination with a carrier, such as a syringe, for carrying the device for performing injections therewith, or is directed to a syringe that may or may not be used with any of the above-described devices. The carrier or syringe includes a syringe body defining a distal end and a proximal end, a device mount located on a distal end of the syringe body and releasably engageable with the device body, a plunger received within a distal end of the syringe body, and a seal engageable with the device body to form a substantially fluid-tight seal therebetween.

In some embodiments of the present invention, the device further comprises a first spring defining a first spring constant and mounted between the needle cap and body for biasing the needle cap and/or body in a direction from a proximal end toward a distal end of the device. The syringe includes a second spring defining a second spring constant and mounted between the plunger and syringe body. The first spring constant is less than the second spring constant to thereby allow the device to move from the first position to the second position prior to injecting fluid from the syringe through the needle of the device.

In some embodiments of the present invention, the syringe defines a compression chamber located between the plunger and syringe body. The syringe body and/or plunger is movable relative to the other between a first position wherein the compression chamber defines a first volume and a second position wherein the compression chamber defines a second volume less than the first volume. The second spring biases the plunger in a direction form the second position toward the first position.

In some embodiments of the present invention, the syringe further comprises a one-way dispensing valve in fluid communication with the compression chamber and defining an outlet valve-opening pressure, and a one-way inlet valve in fluid communication with the compression chamber and defining an inlet valve-opening pressure. Movement of the syringe body and/or plunger in a direction from the first position toward the second position pressurizes fluid in the compression chamber to a pressure at or above the outlet valve-opening pressure to flow through the dispensing valve and the device one-way valve and needle. Movement of the syringe body and/or plunger in a direction from the second position toward the first position opens the syringe inlet valve to allow fluid to flow into the compression chamber while the device one-way valve prevents any fluid within the needle from flowing therethrough and into contact with the syringe.

In some embodiments of the present invention, the compression chamber is defined by a flexible wall located between the dispensing valve and the inlet valve and defining a variable-volume compression chamber. The plunger is slidably received within the syringe body, and the second spring is located between the plunger and syringe body. The force required to move the plunger or syringe body from the first position to the second position is approximately equal to the force required to displace the spring upon moving between the first and second positions. In some such embodiments, the flexible wall is approximately bellows shaped. In some embodiments of the present invention, the one-way dispensing valve includes an elastic valve member, a relatively rigid valve seat, and an axially-elongated, normally-closed valve seam therebetween, and the one-way inlet valve is a flapper valve.

In some embodiments of the present invention, the syringe device mount and/or device body includes a recessed surface and the other of the syringe device mount or device body defines a raised surface receivable within the recessed surface to releasably engage the device to the syringe. In some such embodiments, the syringe device mount or syringe mount defines a tapered connection that is greater than or less than the taper of tapered Luer connections, or defines a thread that is different than the thread of Luer locking connectors.

In accordance with another aspect, the present invention is directed to a device for preventing needle reuse and injury, comprising: a metal needle defining an inlet and an outlet; first means for mounting the metal needle thereon and releasably connecting the device to a syringe; second means movable relative to the first means from (i) a first position for enclosing the metal needle and preventing needle injury; (ii) to a second position for passing or exposing at least a portion of the metal needle outside the second means and penetrating tissue; and (iii) to a third position enclosing the metal needle and locking the first and second means in the third position for preventing needle reuse and injury; and third means in fluid communication with the outlet of the metal needle for allowing liquid or other fluid flow therethrough in a direction from the inlet toward the outlet of the needle and substantially preventing liquid or other fluid flow therethrough in a direction from the outlet toward the inlet of the needle, and for preventing any liquid or fluid within the needle from flowing through the needle inlet and outside the device and cross contaminating. In some embodiments of the present invention, the first means a device body, the second means is a needle cap, and the third means is a one-way valve.

Some embodiments of the present invention further comprise fourth means for biasing one or more of the first means or the second means in a direction from a proximal end toward a distal end of the device. In some embodiments, the one-way valve includes an elastic valve member, a relatively rigid valve seat, and an axially-elongated, normally-closed valve seam therebetween.

Some embodiments of the present invention further comprise fifth means for engaging an opening in a waste or sharps container and for removing the device from a syringe and disposing of same in the waste or sharps container.

In accordance with another aspect, the present invention is directed to any of the above-described devices in combination with a device carrier, such as a syringe, or is directed to a syringe. The carrier or syringe includes a syringe body defining a distal end and a proximal end, sixth means for releasably engaging the first means and releasably mounting the device thereto, seventh means received within a distal end of the syringe body for manually engaging and injecting fluid from the syringe and through the third means and metal needle of the device, and eighth means for forming a substantially fluid-tight seal between the first means and the syringe. Some embodiments further comprise ninth means for allowing the device to move from the first position to the second position prior to injecting fluid from the syringe through the needle of the device.

In accordance with another aspect, the present invention is directed to a method comprising: (i) placing a distal end portion of a needle enclosure in a first position against a subject's skin wherein a metal needle is received within and enclosed by the needle enclosure to prevent needle injury; (ii) pushing the needle against the bias of a first spring from the first position to a second position wherein the metal needle extends through a distal end portion of the needle enclosure and penetrates skin or tissue underlying or in contact with the distal end portion of the needle enclosure; (iii) injecting a dose of substance through a one-way valve in fluid communication with the needle, through the needle and into the skin or tissue; (iv) withdrawing the needle from the skin and allowing the needle enclosure to move under the bias of the spring from the second position to a third position wherein the metal needle is received within and enclosed by the needle enclosure, and the needle enclosure and body are locked in the third position to prevent needle reuse and injury; and (v) substantially preventing liquid flow through the one-way valve in a direction from an outlet toward an inlet of the needle and preventing liquid in the needle from flowing through the needle inlet and outside the device.

In some embodiments of the present invention, the needle enclosure conceals the needle from view in the first and third positions. Preferably, the needle is concealed from view before and during the injection, and more preferably, is concealed from view before, during and following the injection. Some embodiments further comprise removably attaching the device to a carrier, such as a syringe, including, for example, a multiple dose syringe, injecting the dose of substance from the syringe, through the one-way valve and needle, and into the skin or tissue underlying the skin, and after the withdrawing step, removing the device from the syringe and preventing with the one-way valve cross-contamination between the needle and syringe. In some such embodiments, the injecting step includes pushing the syringe against the bias of a second spring to inject the dose of substance, wherein the second spring defines a second spring constant or force required to deflect the second spring that is greater than a spring constant or force required to deflect the first spring.

One advantage of the present invention, and/or of embodiments thereof, is that the device provides both sharps injury prevention (“SIP”) and needle reuse prevention (“RUP”). In the first position, the needle cap encloses the needle and prevents needle or sharps injuries. The spring located between the needle cap and body normally biases the needle cap into the first position. As a result, the needle cannot be moved from the first position to the second position without pressing the distal end of the needle cap against the patient's skin so that the needle can, in turn, be pressed against the bias of the spring, through the distal end of the needle cap, and into the skin. As a result, the needle is prevented from being exposed to, and seen by the patient. This is a significant advantage for patients with needle phobia or who are otherwise frightened by the sight of a syringe needle. When the injection is complete, and the needle is automatically retracted by the spring while the healthcare worker withdraws the device carrier, such as a multiple dose syringe, the needle cap is biased by the spring into the third position enclosing the needle within the cap. In the third position, the needle cap is locked into position enclosing the needle and thus prevents reuse of the needle.

Another advantage of the present invention, and/or of embodiments thereof, is that the intra-needle one-way valve prevents any liquid or other fluid that may be contained within a needle from flowing in a proximal direction through the needle inlet. As a result, any contaminated fluid that may be located within the needle during or following use cannot cross-contaminate the device carrier, such as a multiple dose syringe, and therefore cannot cross-contaminate the next patient or a healthcare worker or other practitioner. In addition, the device can be used with a multiple dose syringe (i.e., the device carrier) where each device with needle is removed from the multiple dose syringe and disposed of after each use, and a new device with needle is attached to the multiple dose syringe for each new patient injection. The one-way valve in the device prevents any cross-contamination from the needle to the multiple dose syringe, and thus prevents cross-contamination from one patient to another or from patient to healthcare worker.

A further advantage of the present invention, and/or of embodiments thereof, is that the multiple dose syringe includes a second spring mounted between the plunger and syringe body that preferably defines a greater spring constant, or requires a greater spring force for deformation of the spring, as compared to the first spring located between the needle cap and body of the device. As a result, the multiple dose syringe does not, or is substantially prevented from injecting a dose of vaccine, medicament or other substance into a patient, until the first spring is compressed, the needle cap is moved from the first position to the second position, and the needle has passed through the distal end of the needle cap and penetrated into the skin and/or tissue of the patient. Once the first spring is so compressed, the second spring is compressed by manually moving the plunger relative to the syringe body to dispense a dose of substance from the syringe, through the one-way valve and needle of the device, and into the patient. Yet another advantage of such multiple dose syringe is that the second spring must be compressed to inject a dose of substance through the needle and into a patient, and thus a substantially constant force (i.e., the force required to compress the spring) is applied from one patient to the next. Thus, in contrast to the prior art as summarized above, the second spring facilitates a substantially constant speed of injection and force applied to the plunger to inject the substance into a patient from one healthcare worker or practitioner to another. This can be a significant advantage where healthcare workers or other practitioners are required to inject numerous patients, such as when vaccinating during or to prevent a pandemic, and can facilitate preventing the difficulties encountered in handling prior art syringes under such circumstances, as summarized above. In contrast to prior art syringes as described above, there is no friction against the inner surface of the syringe, such as caused by the sliding seal between the plunger and barrel of prior art syringes. Rather, movement of the multiple dose actuator or “plunger” of the multiple does syringe is limited only by a spring and flexible bellows, or other flexible wall forming a variable-volume compression chamber, located between the plunger and syringe body.

Other objects and advantages of the present invention, and/or of embodiments thereof, will become more readily apparent in view of the following detailed description of embodiments and accompanying drawings.

### BRIEF DESCRIPTION OF THE DRAWINGS

FIG. 1 is a cross-sectional view of a device embodying the present invention for preventing needle reuse and injury;

FIG. 2 is a side-elevational view of the device of FIG. 1;

FIG. 3 a plan view of the distal end of the device of FIG. 1 including a ring representing an opening to a waste or sharps container superimposed over the end view of the device body to illustrate the manner in which the protuberances on the body allow a used device to be inserted through such an opening, but prevent its removal from the container back through the opening to thereby remove the used device from a syringe and dispose of it in the sharps container without touching it;

FIGS. 4A-4K are a series of perspective, cross-sectional views of the device of FIG. 1 showing the relative movement of the needle cap and device body when moved from the first position (FIGS. 4A-4B) where the metal needle is received within and enclosed by the needle cap to prevent needle injury, to the second position (FIGS. 4C-4H) where the metal needle is passed through the needle opening in the distal end wall of the needle cap to allow the portion of the needle outside the cap to penetrate tissue, to the third position (FIG. 4K) where the metal needle is received within and enclosed by the needle cap, and the needle cap and body are locked in the third position to prevent needle reuse and injury;

FIGS. 5A-5D are a series of perspective views showing (i) the device of FIG. 1 mounted to the distal end of a multiple dose syringe prior to injecting a patient (FIGS. 5A, 5B), (ii) the device pressed against a patient's skin with the needle in the second position penetrating the skin and the multiple dose syringe manually actuated to inject a dose of vaccine, medicament or other substance into the patient (FIG. 5C), and (iii) removal of the device from the distal end of the syringe without touching it by inserting the device through an opening in a sharps container until the protuberances on the body of the device pass through the opening, and then pulling the syringe away from the container opening to allow the protuberances to engage the opening and thereby remove the device from the syringe and retain the used device in the sharps container for disposal as medical waste (FIG. 5D);

FIG. 6A is an upper perspective view of the elastic valve member of the one-way valve of the device of FIG. 1 where the arrows represent fluid flow from the syringe into the inlet formed on the proximal end of the valve member and partially on diametrically opposite sides of the proximal end of the valve member to allow the fluid to flow from the syringe, into the valve inlet and, in turn, into the annular, normally-closed valve seam between the elastic valve member and relatively rigid valve seat;

FIG. 6B is a cross-sectional view of the elastic valve member of FIG. 6A where the arrows represent fluid flow through the valve inlet at the proximal end thereof, movement of the elastic valve member away from the valve seat in response to the fluid pressure exceeding the valve opening pressure to progressively open the normally-closed valve seam as the pressurized fluid moves therethrough, and the passage of fluid through the valve outlet at the distal end of the elastic valve member, where the elastic nature of valve member and the interference fit between the elastic valve member and valve seat prevent fluid flow in the opposite direction to thereby prevent cross-contamination from a used needle;

FIG. 7A is an exploded cross-sectional view of the device of FIG. 1;

FIG. 7B is across-sectional view of the device of FIG. 1;

FIG. 8 is a cross-sectional view of a multiple dose syringe that may be used with the device of FIG. 1;

FIG. 9 is a partially perspective, cross-sectional view of the device of FIG. 1 mounted to the distal end of the multiple dose syringe of FIG. 8;

FIGS. 10A and 10B are a series of pairs of perspective and cross-sectional views of the device of FIG. 1 mounted to the distal end of the multiple dose syringe of FIG. 8 showing the cycle of a patient injection, including (i) application of the distal end of the device to the patient's skin; (ii) partial compression of the needle cap against the bias of its spring as the needle is pushed through the distal end of the needle cap and into the patient's skin; (iii) full compression of the needle cap against the bias of its spring as the needle is pushed fully through the distal end of the needle cap and into the patient's skin and the multiple dose syringe is, in turn, actuated against the bias of its spring to fully compress its spring and inject a dose of vaccine, medicament or other substance from its compression chamber, through the one-way dispensing valve of the syringe, through the one-way valve and needle of the device, and into the patient; (iv) removal of the device and syringe from the patient's skin to thereby allow the needle cap to automatically move under the bias of its spring from the second position to the third position where the metal needle is received within and enclosed by the needle cap, and the needle cap and body are locked in the third position to prevent needle reuse and injury, and to allow the syringe plunger to automatically move under the bias of its spring from the second position back to the first position and thereby pull another dose of vaccine, medicament or other substance through the inlet valve and into the compression chamber to ready the syringe to dispense another dose; and (v) removal of the used device of FIG. 1 (which is locked in its unusable third position) from the distal end of the syringe into a waste or sharps container (FIG. 5D).

FIG. 11A is a partial, cross-sectional view of the syringe of FIG. 8 and a used device of FIG. 1 attached thereto, wherein the used device is partially inserted through the opening in a sharps container for removing the device from the syringe and disposing of it in the sharps container, preferably without touching it;

FIG. 11B is a cross-sectional view of the syringe and device partially inserted through the opening in the sharps container of FIG. 11A;

FIG. 11C is a top plan view of the sharps container of FIG. 11A showing a configuration of the opening for receiving used devices therethrough and removing them from the syringe for disposal;

FIG. 12A is a side elevational view of a plurality devices of the type shown in FIG. 1 provided in sterile condition in a tray and including adhesive backed covers on the proximal ends thereof that can be removed therefrom when ready for use in order to attach the distal end of a multiple dose syringe to each device and, in turn, remove each such device from the tray for use;

FIG. 12B is a top plan view of the devices and tray of FIG. 12 showing that the covers have been removed from some of the devices and the devices removed from the tray for use;

FIG. 13 is a perspective view of the device of FIG. 1 and multiple dose syringe of FIG. 8 where the syringe is connected to a pouch containing a supply of vaccine or other substance, the pouch is received in a container along with one or more cold packs to cool the vaccine in the pouch during use, and a harness including a shoulder strap and/or belt is attached to the container to allow a healthcare worker to carry the container while keeping her hands free and otherwise allow for one-handed vaccinations;

FIG. 14A is a side elevational view of the multiple dose syringe of FIG. 8 wherein the inlet conduit is connected to a single-use sterile connector for sterile connecting the syringe to a source of vaccine, medicament or other substance;

FIG. 14B is a side elevational view of the syringe of FIG. 14A where the single use sterile connector is sterile connected to a pouch containing a supply of vaccine for supplying multiple doses of sterile vaccine through the syringe;

FIG. 15 is a another perspective view of the multiple dose syringe and pouch received within the container with one or more cool packs and provided with a harness for carrying the container and syringe and allowing for one-handed vaccinations;

FIG. 16A is a partial cross-sectional, exploded view of the syringe, single-use sterile connector, pouch, container, cold packs and harness;

FIG. 16B is a view depicting a clinical use of the syringe, single-use sterile connector, pouch, container, cold packs, and harness with the device of FIG. 1;

FIG. 17 is a perspective view of another device embodying the present invention for preventing needle reuse and injury;

FIG. 18 is a cross-sectional perspective view of the device of FIG. 17;

FIG. 19 is cross-sectional side view of the device of FIG. 17;

FIG. 20 is a top perspective view of the cap of the device of FIG. 17;

FIG. 21 is a bottom perspective view of the body of the device of FIG. 17; and

FIG. 22 is a bottom perspective cross-sectional view of the device of FIG. 17.

### DETAILED DESCRIPTION

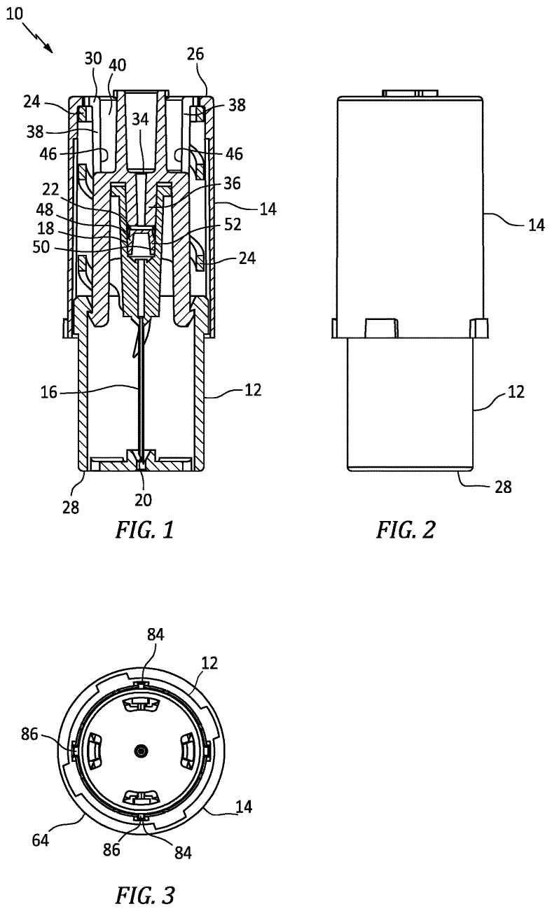

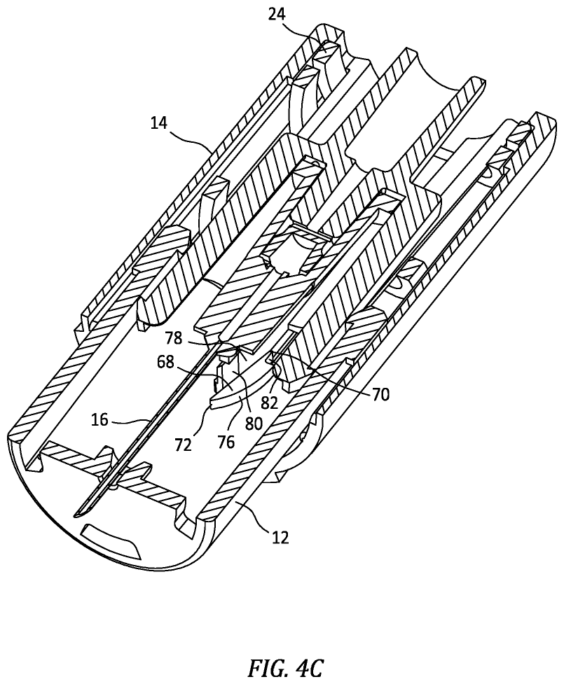

In FIG. 1, a device embodying the invention for preventing needle reuse and injury is indicated generally by the reference numeral 10. The device 10 comprises a needle enclosure or cap 12, a body 14, and a metal needle 16 mounted between the needle cap and body. The metal needle 16 defines an inlet 18 and an outlet 20. The needle cap 12 and/or the body 14 is movable relative to the other from (i) a first position, as shown in FIGS. 4A-4B, wherein the metal needle 16 is received within and enclosed by the needle cap 12 to prevent needle injury; (ii) to a second position, as shown in FIGS. 4C-4H, wherein at least a portion of the metal needle 16 is passed through, exposed or exposable outside the needle cap 12 to allow the portion of the needle 12 outside the cap to penetrate tissue; and (iii) to a third position, as shown in FIG. 4K, wherein the metal needle 16 is received within and enclosed by the needle cap 12, and the needle cap 12 and body 16 are locked in the third position to prevent needle reuse and injury.

The device 10 further includes a one-way valve 22 in fluid communication with the inlet 18 and outlet 20 of the metal needle 16. The one-way valve 22 allows liquid or other fluid flow therethrough in a direction from the inlet 18 toward the outlet 20 of the needle 16, and substantially prevents liquid or other fluid flow therethrough in a direction from the outlet 20 toward the inlet 18 of the needle 16, to thereby prevent any liquid or other fluid within the needle from cross contaminating, such as by flowing through the needle inlet and outside the device 10.

A first spring 24 is mounted between the needle cap 12 and body 14 and biases the needle cap in a direction from a proximal end 26 toward a distal end 28 of the device 10. In the illustrated embodiment, the first spring 24 is formed integral with the needle cap 12 and is substantially coil-shaped. However, as may be recognized by those of ordinary skill in the pertinent art based on the teachings herein, the first spring 24 may be formed separate from the needle cap 12, and may take any of numerous different configurations and may be formed of any of numerous different materials that are currently known, or that later become known. The first spring 24 may be composed of any suitable material, such as, for example, but not limited to, metal or plastic.

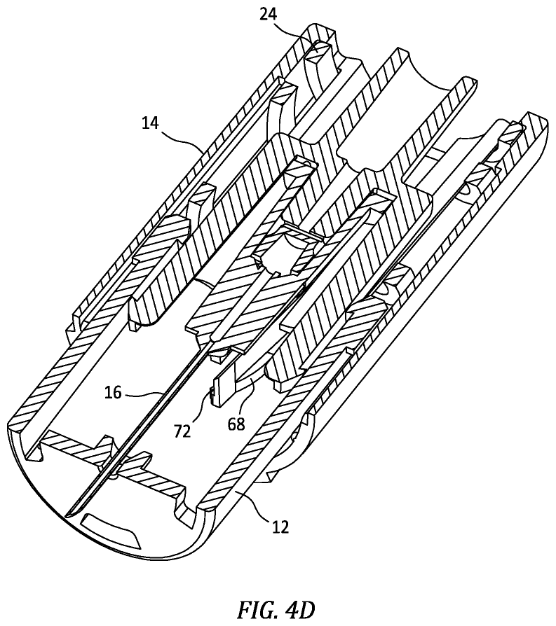

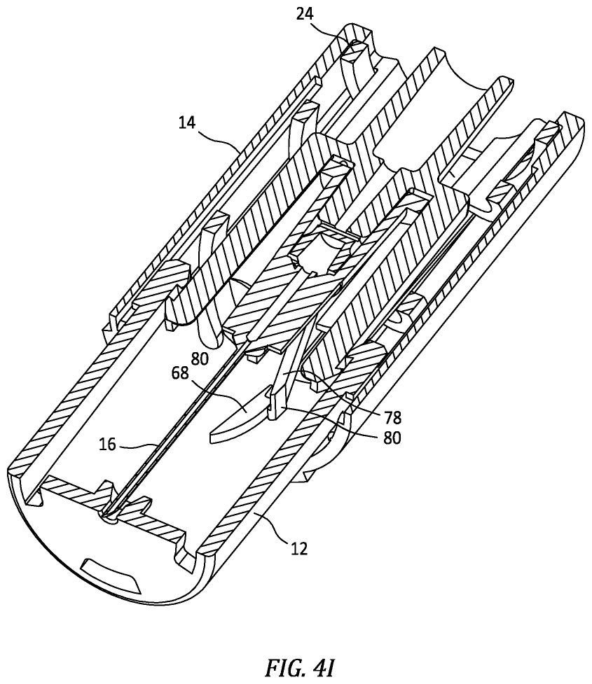

The body 14 includes a syringe mount 30 configured for releasably mounting the device 10 to a syringe 32 (FIGS. 5A-5D). The syringe mount 30 includes an inlet port 34 in fluid communication with the one-way valve 22 to allow fluid to flow from the syringe 32 connected thereto to the one-way valve. The syringe mount 30 further includes a needle support 36 which includes the base of the needle 16 mounted thereto and defines the inlet port 34 therethrough. A syringe mounting flange 38 is radially spaced relative to the needle support 36 and defines a gap 40 therebetween for receiving a distal end portion 42 of the syringe 32 and releasably mounting the device 10 thereto. The syringe mounting flange 38 is configured to releasably mount the device 10 to the syringe 32. As shown in FIG. 8, the syringe 32 includes on the distal end portion 42 thereof a recessed annular surface or groove 44. As shown in FIG. 1, the syringe mounting flange 38 includes a raised annular surface or protuberance 46 that is engageable with the recessed annular surface 44 of the syringe to releasably mount the device 10 to the syringe 32. In the illustrated embodiment, the annular protuberance 46 of the device 10 is snap fit into the annular recess 44 on the syringe to thereby releasably attach the device 10 to the syringe 32.

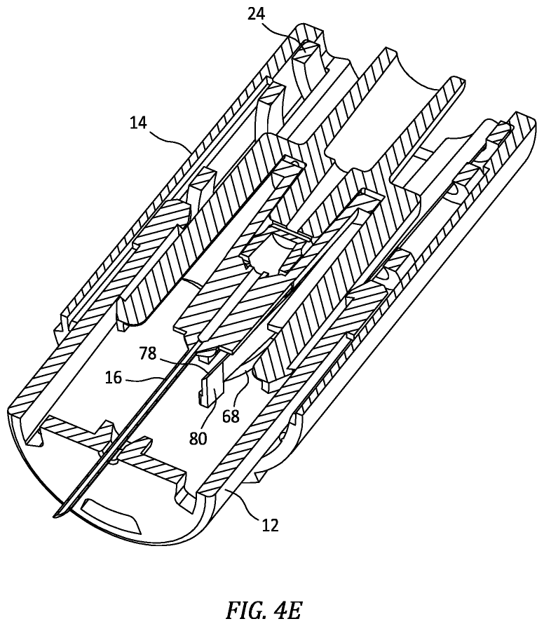

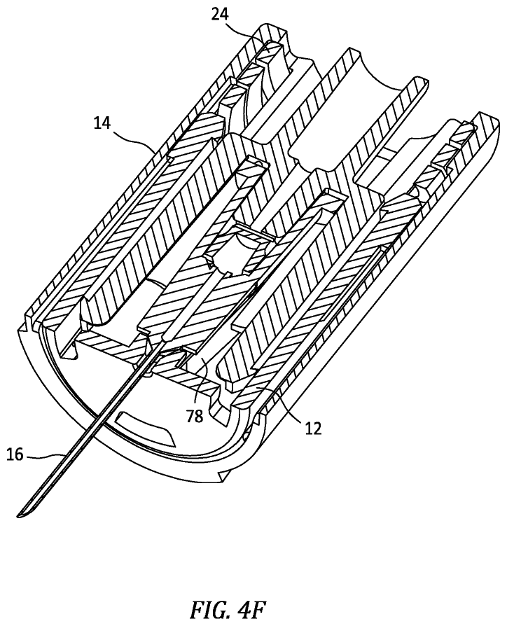

As shown in FIGS. 1, 6A, 6B and 7A, the one-way valve 22 includes an elastic valve member 48, a relatively rigid valve seat 50, and an axially-elongated, normally-closed valve seam 52 therebetween. The elastic valve member 48 defines an interference fit with the valve seat 50, a valve inlet 54 located at a proximal end of the valve seam 52, and a valve outlet 56 located at a distal end of the valve seam. The one-way valve 22 defines a valve-opening pressure based on the degree of interference between the elastic valve member 48 and valve seat 50. In the illustrated embodiment, the valve seat 50 is defined by an annular surface, and the elastic valve member 48 is received within the annular surface and forms the interference fit therebetween. Also in the illustrated embodiment, the annular surface of the valve seat 50 tapers inwardly in a direction from the inlet 54 toward the outlet 56 of the one-way valve. The needle 16 includes a hollow base 58, and the elastic valve member 48 is received therein between the valve seat 50 and the distal end of the needle mount 36. The elastic valve member 48 is compressed into engagement with the valve seat 50 and distal end of the needle mount 36. The elastic valve member 48 defines on a proximal end thereof a recessed surface 60 defining an inlet port in fluid communication between the normally-closed, axially-elongated valve seam 52 and the fluid passageway 34 in the needle mount 36. As can be seen, the elastic valve member 48 forms a fluid-tight seal against the distal end of the needle mount 36. In the illustrated embodiment, the elastic valve member 48 is substantially cylindrical shaped, defining a hollow interior to facilitate molding and flexing of the annular wall thereof under fluid pressure to progressively open the normally closed valve seam 52 in response to fluid exceeding the valve-opening pressure and to progressively close the seam upon passage of the fluid therethrough. The valve inlet 60 is defined by a groove extending across the proximal end wall of the valve member 48 and partially along the annular side wall of the valve member at the proximal end thereof to direct the pressurized fluid into the normally-closed, axially extending valve seam 52 on diametrically opposite sides of the valve member.

One advantage of the valve member 48 is that it is configured to fit within the hollow base 58 of the needle 16 and form the one-way valve therebetween. In the illustrated embodiment, the needle 16 is a commercially available needle, such as a conventional needle manufactured by Becton, Dickinson and Company or BD, wherein the needle is metal and the plastic base 58 forms a female Luer connector. As shown in FIG. 1, the distal end of the needle mount 36 forms a male Luer connector to mount the base 58 of the needle to the needle mount 36 with the elastic valve member 48 compressed therebetween. The connection between the needle base 58 and needle mount 36 is preferably a standard or conventional connector, such as a Luer slip connector or a Luer lock connector, to allow the device 10 to use conventional, commercially-available needles. However, as may be recognized by those of ordinary skill in the pertinent art based on the teachings herein, the needle and needle mount may take any of numerous different configurations and/or may be formed of any of numerous different materials that are currently known, or that later become known.

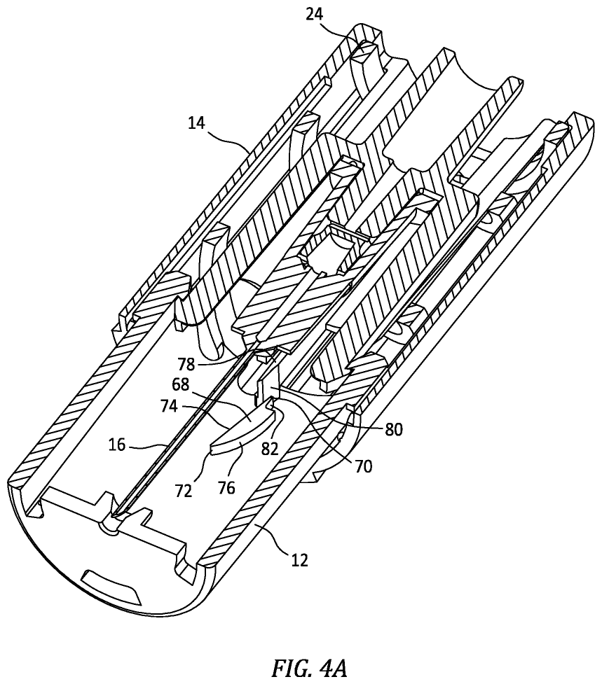

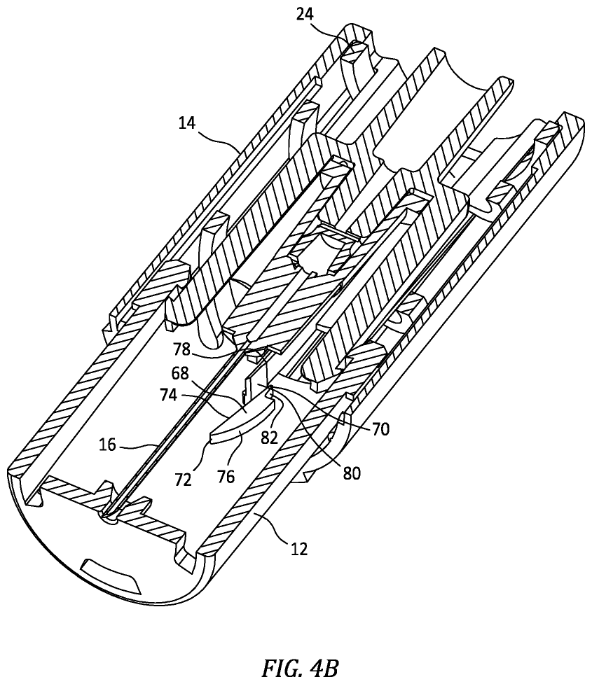

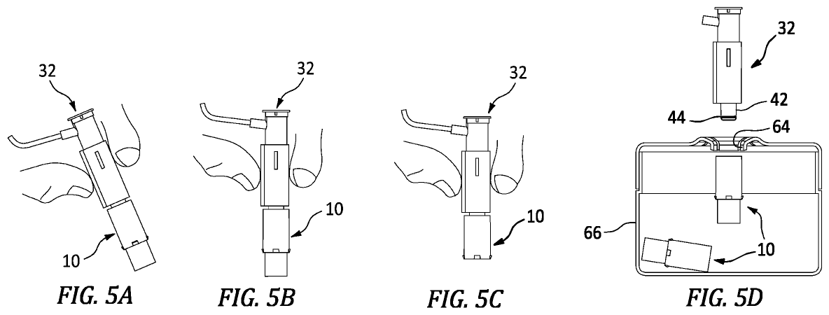

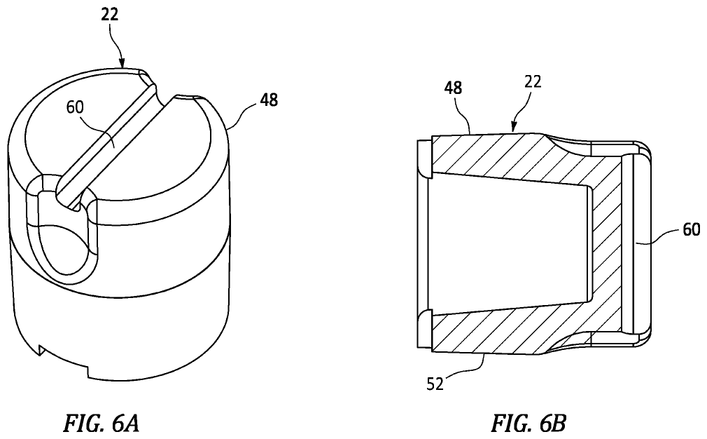

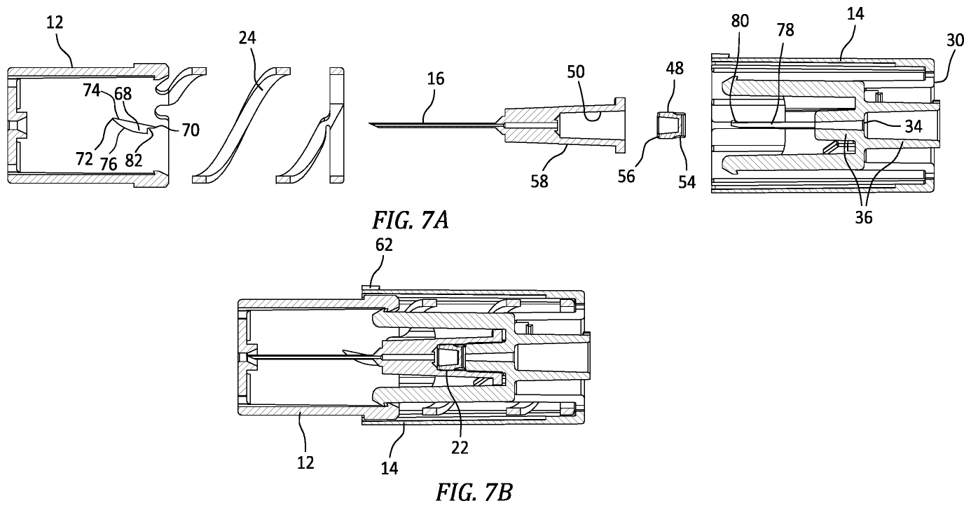

As shown in FIGS. 1-3, the body 14 includes on an exterior surface thereof protuberances 62, 62 engageable with an opening 64 in a waste or sharps container 66 (FIGS. 5D, 11A-C) to remove the used device 10 from the syringe 32 and dispose of same in the container. The protuberances 62, 62 are located adjacent to the proximal end 26 of the body 14 on diametrically opposite sides of the body relative to each other. Each protuberance 62, 62 defines a curvilinear, exterior surface that tapers outwardly in a direction from the distal end toward the proximal end of the body. Accordingly, as shown in FIGS. 3 and 5D, the tapered exterior surfaces of the protuberances 62, 62 allow them to be pushed through the opening 64 into the sharps container 66 but prevent the device from being pulled in the opposite direction through the container opening. Rather, the proximal ends of the protuberances 62, 62 engage the underside of the peripheral edge of the opening 64 of the container 66 to prevent movement of the device back through the opening. As the user continues to pull the syringe 32 away from the container opening 64, the distal end portion 42 is disconnected from the device 10 and the device is, in turn, dropped into the waste or sharps container (FIG. 5D) for subsequent disposal as medical waste.

As shown in FIGS. 4A-4K, the needle cap 12 includes a locking member 68 on an interior surface thereof defining a proximal end 70, a distal end 72, a first side 74 located between the proximal and distal ends, and a second side 76 opposite the first side and located between the proximal and distal ends. The body 14 includes a locking arm 78. The locking arm 78 slidably contacts the first side 74 of the locking member 68 during movement from the first positon (FIG. 4A) to the second position (FIG. 4F), and slidably contacts the second side 76 of the locking member during movement from the second position (FIG. 4F) to the third position (FIG. 4K). The locking arm 78 is biased into contact with the locking member 68. The locking arm 78 defines a distal end 80 that slidably contacts the locking member 68. As shown in FIGS. 4A-4K, the locking arm 78 flexes laterally while it engages and slidably contacts the locking member 68 during movement from the first positon to the second position, and from the second position to the third position. The second side 76 of the locking member 68 defines a recess 82 at the proximal end 70 thereof. In the third position (FIG. 4K), the distal end 80 of the locking arm 78 is biased into the recess 82 of the locking member 68 to thereby lock the needle cap 12 and body 14 in the third position (FIG. 4K). As set forth above, the distal end 80 of the locking arm 78 is flexed into engagement with the locking member 68, and in the third position (FIG. 4K), the distal end 80 of the locking arm 78 is snapped into engagement with the recess 82 of the locking member 68 to thereby lock the needle cap 12 and body 14 in the third position (FIG. 4K). As shown in FIGS. 4A-4K, the first and second sides 74 and 76, respectively, of the locking member 68 are oriented at one or more acute angles relative to each other. In the illustrated embodiment, the first side 74 is approximately planar, whereas the second side 76 is curvilinear. As may be recognized by those of ordinary skill in the pertinent art based on the teachings herein, the locking member and locking arm may take any of numerous different configurations, and the device may include any number of such components or different components that are currently known, or that later become known, for performing one or more of the functions thereof as set forth herein.

As shown in FIG. 3, the body 14 defines a plurality of axially-extending recessed surfaces or grooves 84, 84 and the needle cap 12 defines a corresponding plurality of axially-extending raised surfaces or ribs 86, 86 received within the recessed surfaces or grooves and preventing rotation of the needle cap and/or body relative to the other. In the illustrated embodiment, the needle cap 12 defines four, equally-spaced ribs 86, 86 and the body 14 defines four, equally-spaced axially-extending grooves 84, 84 that receive the respective ribs therein. As may be recognized by those of ordinary skill in the pertinent art based on the teachings herein, the device may include any number of recessed surfaces and corresponding raised surfaces, and such surfaces may take any of numerous different configurations, or the device may include alternative features, that are currently known, or that later become known, for performing the function of such features as set forth herein.

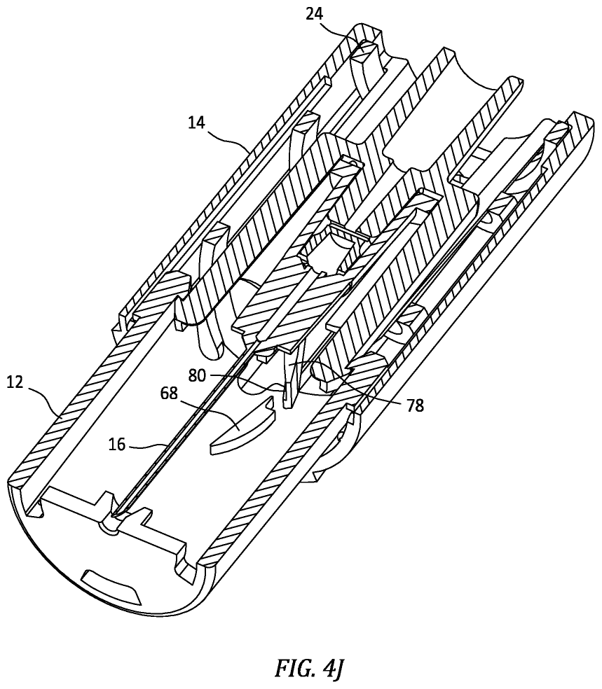

As shown in FIG. 8, the syringe 32 includes a syringe body or barrel 88 defining a distal end 90 and a proximal end 92. The device mount 42 is located on the distal end 90 of the syringe body 88 and, as set forth above, is releasably engageable with the device body 14. A syringe actuator or plunger 94 is received within the proximal end 92 of the syringe body 88 and a seal 96 is engageable with the device body 14 to form a substantially fluid-tight seal therebetween (FIG. 9).

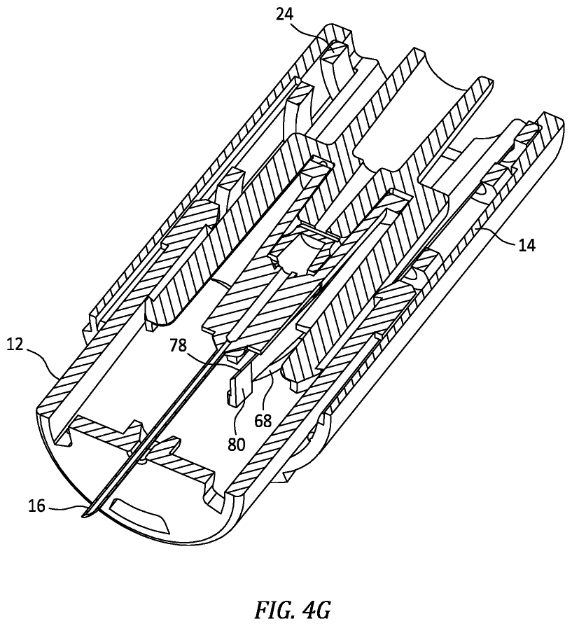

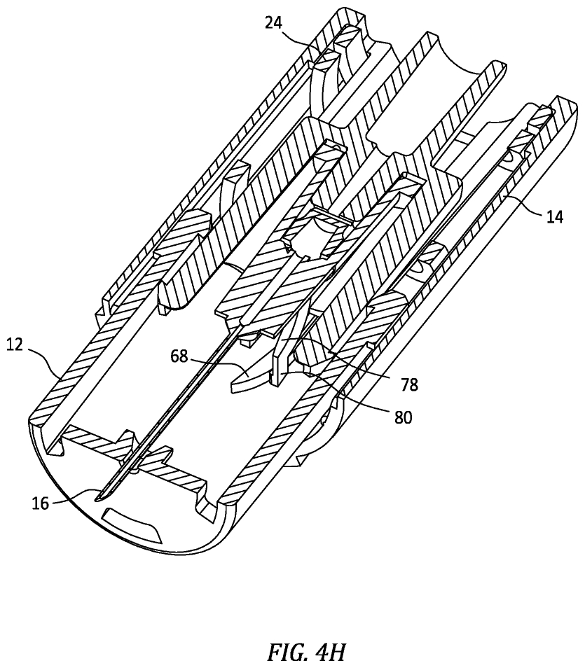

The syringe 32 includes a second spring 98 defining a second spring constant and mounted between the plunger 94 and syringe body 88. The spring constant of the spring 24 of the device 10 (FIG. 7A) is less than the second spring constant to thereby allow the device 10 to move from the first or normal position (FIG. 4A) to the second or compressed position (FIG. 4F) prior to injecting fluid from the syringe 32 through the needle 16 of the device 10.

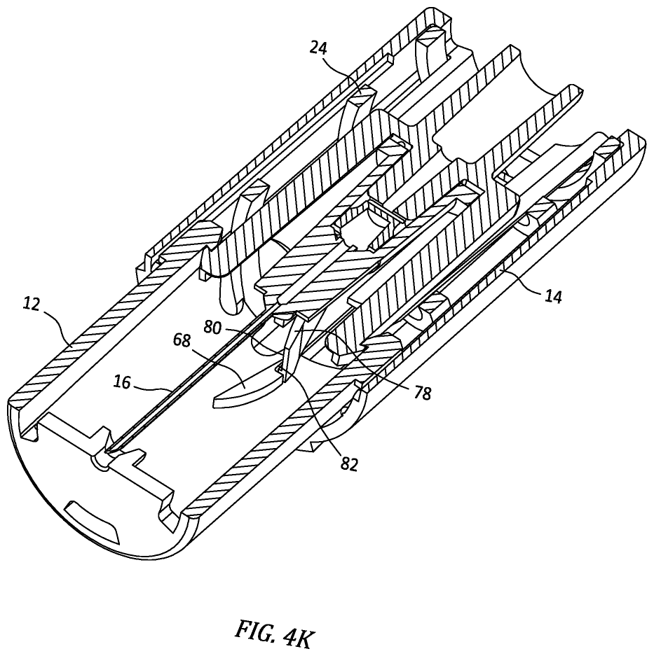

As shown in FIG. 8, the syringe 32 defines a compression chamber 100 located between the plunger 94 and syringe body 88. The syringe 32 is actuated by manually moving the actuator or plunger 94 relative to the syringe body 88 between a first or normal position (FIG. 8) wherein the compression chamber defines a first volume and a second compressed or actuated position (FIG. 10A) wherein the compression chamber defines a second volume less than the first volume. The second spring 98 biases the plunger in a direction form the second position toward the first position, i.e., in a direction from the distal end 90 toward the proximal end 92 of the syringe body.

The syringe 32 further comprises a one-way dispensing valve 102 in fluid communication with the compression chamber 100 and defining an outlet valve-opening pressure, and a one-way inlet valve 104 in fluid communication with the compression chamber 100 and defining an inlet valve-opening pressure. Movement of the syringe body 88 and/or plunger 94 in a direction from the first or normal position (FIG. 8) toward the second or compressed position (FIG. 10A), i.e., in a direction from the proximal end toward the distal end, pressurizes fluid in the compression chamber 100 to a pressure at or above the outlet valve-opening pressure to flow through the dispensing valve 102 and the device one-way valve 22 and needle 16. Movement of the syringe body 88 and/or plunger 94 in a direction from the second or compressed position (FIG. 10A) toward the first or normal position (FIG. 8), i.e., in a direction from the distal end toward the proximal end, opens the syringe inlet valve 104 to allow fluid to flow into the compression chamber 100, while the device one-way valve 22 prevents any fluid within the needle 16 from flowing therethrough and into contact with the syringe. The spring constant or force of the spring 98 is preferably sufficient to automatically move the plunger 94 from the second or compressed position back into the first or normal position.

The compression chamber 100 is defined by a flexible wall 106 located between the dispensing valve 102 and the inlet valve 104 and defining a variable-volume compression chamber 100. In the illustrated embodiment, the flexible wall 106 is approximately bellows shaped. The plunger 94 is received within the syringe body 88, and the second spring 98 is located between the plunger 94 and syringe body 88. The force required to move the plunger 94 or syringe body 88 from the first or normal position (FIG. 8) to the second or compressed position (FIG. 10A) is approximately equal to the force required to displace the spring 98 upon moving between the first and second positions. As shown in FIG. 9, the bellows 106 defines the variable-volume compression chamber 100, and thus this design obviates the need for a plunger with an elastic seal that slidably engages a syringe barrel, as encountered in the above-described prior art. Accordingly, the actuator or plunger 94 can be actuated numerous times as may be required to deliver multiple doses from the syringe 32 through respective devices 10 without generating undesirable particles that otherwise could result from a sliding elastic seal as encountered in the above-described prior art. Yet another advantage is that because there is substantially zero or negligible friction force between the actuator/plunger 94 and body 88, the force required to actuate the syringe and inject a dose is approximately equal to the force required to compress the spring 98 upon moving the actuator/plunger from the first to the second position. A further advantage of the illustrated embodiment is that the compression chamber 100 cannot be opened as in prior art syringes. Rather, the compression chamber is sealed within the bellows 106 located between the plunger and body. Preferably, as shown, the bellows 106 is injection molded in one piece with the elastic dispensing valve member 102 and annular seal 96, and this part is co-molded with the plunger 94. As a result, a user cannot open the device and access the compression chamber without visibly damaging or destroying it.

The one-way dispensing valve 102 includes an elastic valve member 108, a relatively rigid valve seat 110, and an annular, axially-elongated, normally-closed valve seam 112 therebetween. The elastic valve member 108 surrounds and receives the relatively rigid valve seat 110, and the outer diameter of the valve seat is greater than then inner diameter of the elastic valve member to form an interference fit, and thus the normally-closed, axially extending seam therebetween. In the illustrated embodiment, the elastic valve member 108 tapers inwardly in a direction from the proximal end toward the distal end of the valve. Accordingly, progressively less force is required to open the valve as the fluid moves from the proximal end toward the distal end of the valve seam, and thus the valve inherently forces fluid within the valve seam to be dispensed therefrom. As shown in FIG. 8, the valve seat 110 defines on the distal end thereof a recess, which in the illustrated embodiment is substantially hemi-spherical or concave shaped, and extends diametrically across substantially the entirety of the distal face from approximately one side of the valve seam 112 to the diametrically opposite side of the valve seam. In the event that any residual liquid collects on the distal end of the valve seam 112, the residue is drawn or pulled away from the distal end of the valve seam and into the recessed tip, such as by capillary action, to thereby prevent such residue from intermixing with subsequent doses injected from the dispensing valve 102 into a device 10.

As shown in FIG. 8, the annular seal 96 is formed by the base of the elastic valve member 108 and protrudes distally from the base wall of the connecting end portion 42 of the syringe. Thus, as shown in FIG. 9, when the device 10 is releasably connected to the distal end portion 42 of the syringe 32, the proximal end 112 of the syringe mount 30 engages the seal 96 to form a fluid-tight annular seal therebetween. As also shown in FIG. 9, when the device 10 is connected to the syringe 32, the dispensing valve 102 of the syringe is received with a hollow inlet cavity or well 114 formed in the proximal end of the syringe mount 30. As indicated by the arrows in FIG. 9, movement of the syringe plunger 94 against the bias of the second spring 98 from the first or normal position to the compressed or second position, pressurizes fluid within the compression chamber 100 above the dispensing valve opening pressure to, in turn, dispense fluid through the dispensing valve 102, into the well 114 and inlet 34 of the syringe mount 30, through the one-way valve 22 and needle 16, and into a patient. As shown in FIG. 9, there is sufficient annular space between the exterior of the elastic valve member 108 and interior of the well 114 to allow the elastic valve member to expand and open in response to fluid at its inlet exceeding its valve-opening pressure. The fluid-tight seals formed by the seal 98 at the distal end of the syringe 32 and by the elastic valve member 48 in the device 10 facilitate maintaining sufficient fluid pressure upon dispensing fluid from the dispensing valve 102 into the device 10 to overcome the valve-opening pressure of the one-way valve 22 of the device 10 and thereby inject the fluid through the needle 16 and into a patient.

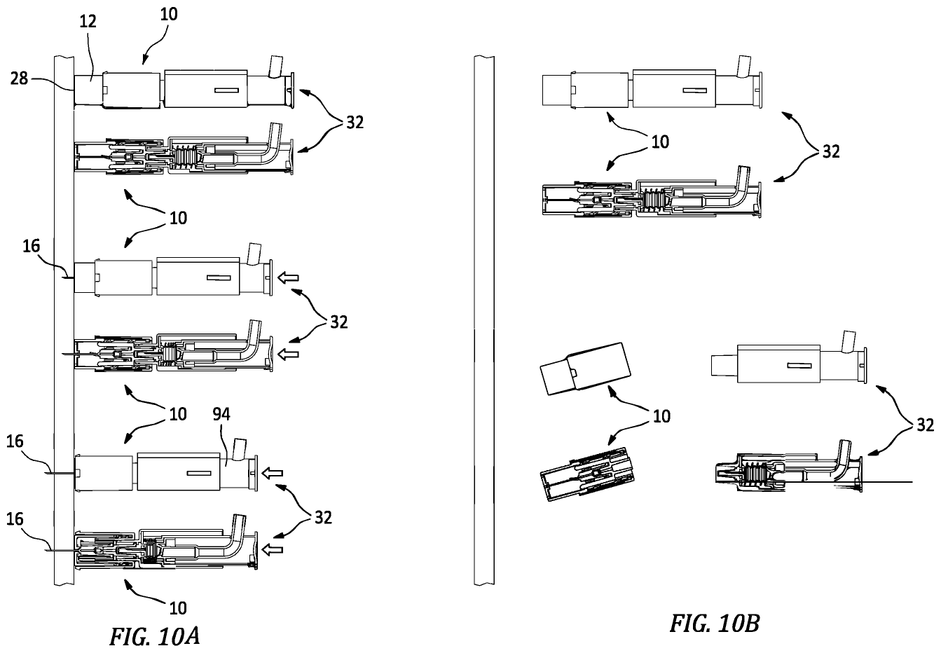

In the illustrated embodiment, the one-way inlet valve 104 to the compression chamber 100 is a flapper valve defining a flexible flap overlying a valve inlet or aperture formed in a relatively rigid valve seat. Upon moving the plunger 94 against the bias of the second spring 98 from the first or normal position to the second or compressed position, the fluid pressure within the compression chamber 100 forces the flap into the closed position, whereas movement of the plunger in the opposite direction under the force of the second spring 98 pulls the flap away from the valve seat to open the valve and pull another dose of vaccine, medicament or other substance into the compression chamber. As shown in FIG. 8, the inlet valve 104 is connected in fluid communication with a distal end of an inlet conduit 116. The proximal end of the inlet conduit 116 is connected in fluid communication with a pouch (FIGS. 13-16) containing the source of vaccine, medicament or other substances. As may be recognized by those of ordinary skill in the pertinent art based on the teachings herein, the dispensing valve, inlet valve and pouch or other storage chamber or source of substance, may take any of numerous different configurations that are currently known, or that later become known.

As indicated above and shown in FIG. 9, the syringe device mount 42 includes a recessed surface or groove 44 and the syringe mount 30 defines a raised surface or rib 46 receivable within the recessed groove to releasably engage the device 10 to the syringe 30. In the illustrated embodiments, the syringe device mount 42 is not a Luer connection and therefore does not allow connection thereto of a conventional needle, such as a needle with Luer connector. One advance of this configuration is that the syringe only allows connection of the device 10 thereto and therefore does not allow for needle reuse and prevents sharps injuries for this additional reason. If the syringe mount 42 were to define a tapered or threaded connection, preferably the taper would be different, i.e., greater than or less than, the taper of tapered Luer connections, or the thread would be different than the threads of Luer locking connectors. As a result, the conventional Luer connectors would not be connectable to the syringe 32 to thereby further prevent needle reuse or sharps injuries.

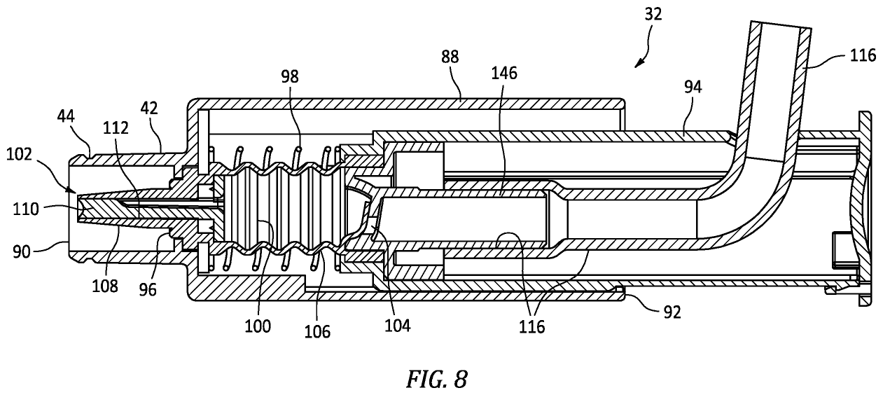

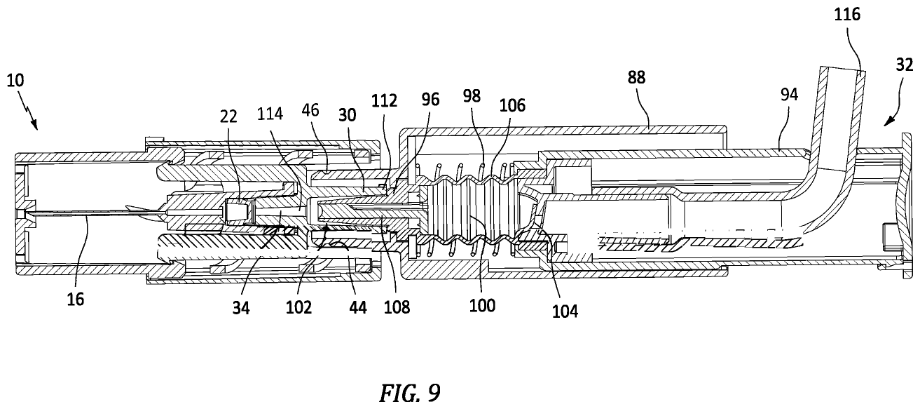

As shown in FIGS. 12A and 12B, a plurality of devices 10 may be provided in sterile condition in a multiple device carrier or tray 118 including a plurality of receptacles 120, wherein each receptacle is configured to receive a respective device 10 therein with its proximal end 26 facing up. An adhesive-backed cover 122 overlies and is removably attached to the proximal end 26 of each device 10 in order to maintain the interior of the device sterile when located in the tray. As shown in FIG. 12B, each cover 122 includes a manually-engageable tab 124 to allow the cover to be gripped and peeled away from the proximal end of the respective device 10 when ready for use. As shown in FIG. 12A, when the cover 124 is removed, the distal end portion 42 of the syringe is inserted into the syringe mount 30 on the proximal end of the device 10 to releasably connect the device to the syringe and remove the device from its respective receptacle in the tray for use. One advantage of this configuration is that it allows single-handed connection of the devices 10 to the multiple dose syringe 32 and, in turn, single-handed removal of the used devices from the syringe and disposal of the used devices, such as into a sharps container, as shown in FIGS. 5D and 11C. As shown in FIGS. 11A and 11B, the opening 64 in the sharps container 66 preferably allows for itself to be flexed inwardly when pushing a device 10 and its lateral protuberances 62, 62 therethrough, but that resists upwardly flexing of the opening to prevent a used device 10 from being pulled out of the opening and otherwise to facilitate removing the used devices 10 from the syringe 32. As shown in FIG. 11A, the opening 64 may define a reinforced edge 126 that defines an increased cross-sectional thickness on the underside of the opening, and is progressively thicker when moving radially inwardly along the edge. In addition, the opening 64 may include a plurality of radially-extending slots 128, 128 to facilitate inward flexing of the opening edge 126 when inserting devices 10 therethrough. The illustrated embodiment includes four radially-extending slots 128, 128 approximately equally spaced relative to each other. However, as may be recognized by those of ordinary skill in the pertinent art based on the teachings herein, any number of slots or other features may be used to form the sharps container and otherwise facilitate removal of the devices from the syringe, preferably allowing for one-handed removal and/or without touching them. One advantage of the device 10 and waste container 66 is that the device 10 is disconnected from the syringe 32 without contact between the syringe 32 and waste container, thereby preventing contamination of the syringe by the waste container. Yet another advantage is that the disclosed configuration allows disconnection and disposal of the device 10 without touching of the device by a healthcare worker. In other words, the healthcare worker need only handle the syringe 32 while inserting the device 10 releasably attached thereto into the opening 64 of the waste container 66 to remove the device from the syringe and dispose of it in the waste container.

As my be recognized by those of ordinary skill in the pertinent art based on the teachings herein, numerous changes may be made to the device carrier or tray, the sharps container, and the methods or apparatus for supplying, holding and disposing of the devices, that are currently known or that later become known. For example, the device carrier may include any number of receptacles or other features for holding any number of devices. In one such exemplary embodiment, each device carrier includes a single receptacle for holding a respective device. In other embodiments, the device carrier includes multiple receptacles or other features for holding multiple devices. In addition, the device carrier can be used to both supply the devices and dispose of them. In one such embodiment, the used devices 10 are inserted back into the receptacle and snap-fit or otherwise secured to the carrier to allow disconnection of each used device from a syringe without requiring a user to touch the device. In one such embodiment, the receptacle(s) in the carrier define protuberances or bumps that extend radially inwardly and are aligned with the protuberances 62, 62 on the body 14 of the device 10 and are configured to allow the used device 10 to be inserted into the receptacle but prevent the used device from being removed from the receptacle. The receptacle also may define one or more grooves or other features to substantially prevent rotation of the device 10 within or relative to the receptacle of the device carrier. Once the used device 10 is received in the receptacle of the device carrier, the syringe 32 may be pulled away from the used device and/or rotated relative to the used device to disconnect the syringe from the used device, dispose of the used device, and reattach the syringe to a different new or unused device 10. If the device carrier holds only a single device 10, the device carrier can be used to safely receive, hold and dispose of the used device 10. If, on the other hand, the device carrier holds multiple devices 10, the device carrier can collect and hold a plurality of used devices 10, and when filled with the plurality of used devices 10, the carrier filled with the used devices 10 can be disposed of One advantage of a device carrier that holds multiple devices 10 is that it facilitates eliminating the need for a healthcare worker to hold the device carrier while inserting a used device 10 therein, and thus facilitates eliminating the possibility of the healthcare working touching the used device 10.

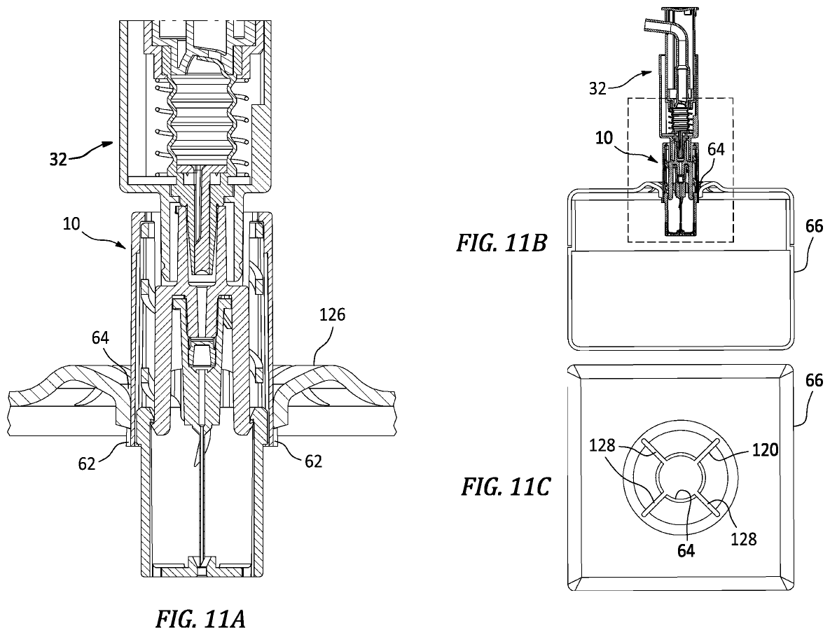

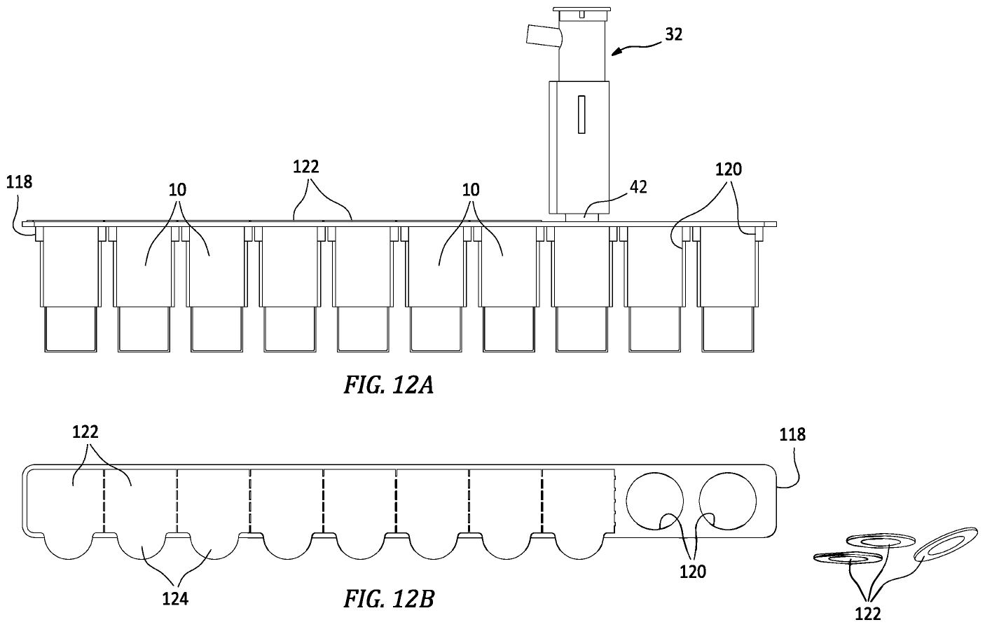

As shown in FIGS. 13-16, the multiple dose syringe 32 may be connected in fluid communication with a multiple dose pouch 130 containing multiple doses of sterile vaccine, medicament or other substance. As shown in FIGS. 14A and 14B, the proximal end of the inlet conduit 116 of the multiple dose syringe 32 is connected to a single-use, sterile connector 132. The pouch 130 includes a filling port 134 for sterile filling the pouch and resealing the port after filling, and a dispensing port 136 for connecting the sterile, single-use connector 132 thereto. Each port 134, 136 includes a penetrable septum allowing the filling port 134 to be needle penetrated and filled therethrough, and allowing the dispensing port 136 to be sterile connected to the interior of the pouch by penetrating the respective septum with a needle or like member on the connector. Once the pouch 130 is sterile connected to the multiple does syringe 32, multiple doses of vaccine, medicament or other substance may be dispensed from the pouch and sterile injected into patients. Each patient injection uses a respective device 10 to prevent needle reuse and sharps injuries, as described above.

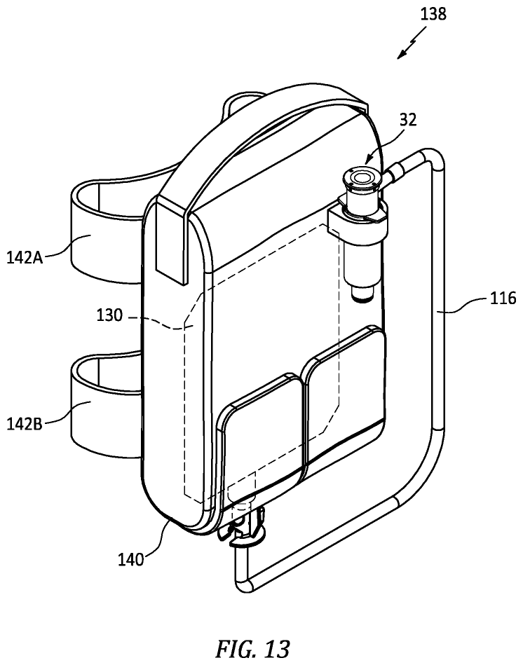

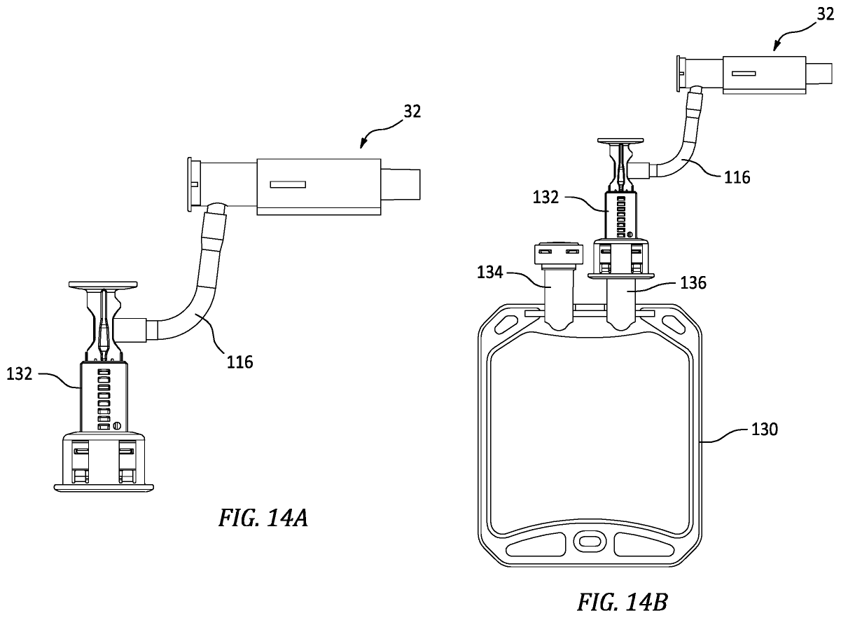

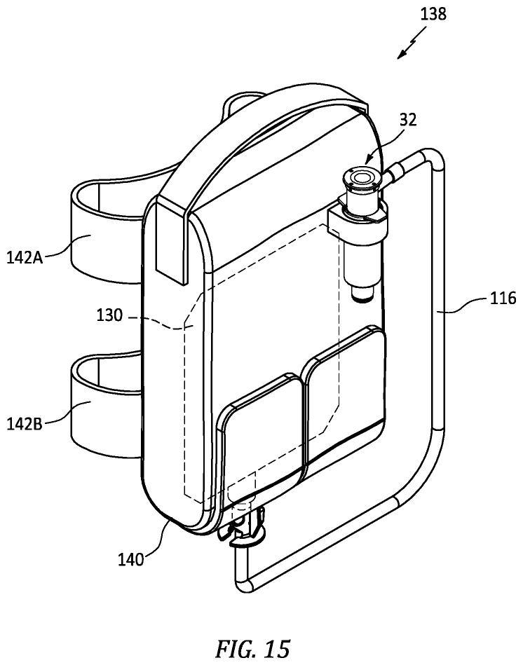

As shown in FIGS. 13 and 15, a harness 138 and container 140 may be provided to carry multiple doses of vaccines or other substances, and facilitate use of the syringe 32 and devices 10 on multiple patients. The pouch 130 is received within the container 140, and the container includes an opening, such as in a bottom wall thereof, to allow the inlet conduit 116 of the multiple dose syringe 32 to pass therethrough. The harness 138 includes one or more straps 142 including a first or shoulder strap 142A and a second strap or belt 142B. The shoulder strap 142A fits over a person's shoulder and the belt 142B fits around a person's waist or torso in order to carry the container 140 and pouch 130 therein about the person's waste or torso. As shown in FIG. 16, the container 140 may be sized to fit the pouch 130 along with one or more cold packs 144, 144 therein. In the illustrated embodiment, the container 140 is sized to fit a pouch 130 along with two cold packs 144, 144 packed within the container on opposite sides of the pouch relative to each other. This configuration facilitates maintaining a vaccine, for example, or other substance, below a required temperature while dispensing multiple doses of the vaccine or other substance to multiple patients. As shown in FIG. 16, rather than include a shoulder strap, the container may include a necklace or neck strap 142C that fits over a healthcare worker's head and neck, and a torso strap 142B that fits around the torso in order to carry the device and free the healthcare worker's hands to hold the syringe 32 and otherwise perform vaccinations or other injections.

As may be recognized by those of ordinary skill in the pertinent art based on the teachings herein, other combinations of straps, strap configurations, and/or harnesses, and other types of containers, that are currently known or that later become known, equally may be employed. For example, the harness or straps may be configured to hang or otherwise support the container from a shoulder, arm, wrist, thigh, back or other body part, or to hang or otherwise support the container from a stand, table or other structure. In some embodiments, for example, one or more armbands is utilized in place of the harness 138 to secure the container 140 or pouch 130 to the arm, e.g., the upper arm, of a user. Use of an armband may locate the pouch 130 at a greater height relative to the height of a patient's shoulder, which helps keep any air in the pouch 130 and prevents air from flowing into the syringe 130. This greater height also reduces the change that, if a user drops the syringe 130, that the syringe will contact the floor or other surface and become contaminated. Further, with the pouch 130 located on the user's arm, the user can more easily view the pouch 130 and see that is empty or nearly empty, and possibly pumping air into the conduit 116, stop pumping doses, and change out the pouch for a new (full) one.

In addition, a syringe mount, such as a cradle, may be provided to releasably mount the syringe thereto when not in use. In one such embodiment, the syringe mount or cradle is located on an exterior of the container for holding the pouch, or a frame is provided to hold the container and/or pouch, and the syringe mount or cradle is formed or otherwise mounted thereon. In another embodiment, the syringe mount or cradle is configured to be mounted on a person, such as on a healthcare workers' hand, wrist or arm. In one such embodiment, the syringe mount or cradle includes one or more respective straps or other structures configured to strap or releasably attach the cradle to a healthcare worker's hand, wrist or forearm. The container with pouch, on the other hand, is strapped or otherwise releasably attached to the healthcare worker's arm or back, such as by one or more arm and/or shoulder straps or in a backpack. In one such embodiment, the container is supplied in a backpack and the syringe mount or cradle is strapped to a wrist or forearm. In another embodiment, the container with pouch is strapped to the upper arm, and the syringe mount or cradle is strapped to the wrist or forearm of the same arm. In each such case, the conduit connected between the syringe and pouch is sufficiently long to allow the syringe to be held and used to make injections with either hand. The straps can include Velcro fasteners (i.e., interlocking hook and loop tapes or other such fasteners or closures), or other types of fasteners, clasps or closures, to facilitate wrapping the straps around the body parts or otherwise attaching them thereto, and releasably securing the straps or syringe mount or cradle in place. The syringe mount or cradle can include one or more recessed surfaces for receiving the syringe and other surfaces or fittings for snap fitting or otherwise releasably attaching the syringe thereto.

Once a device 10 is removably attached to the multiple dose syringe 32, the needle cap 12 and body 14 are located in the first position, as shown in FIG. 4A, where the metal needle 16 is received within and enclosed by the needle cap 12 to prevent needle injury and conceal the metal needle from view. Then, as shown in FIG. 10A, in order to inject a dose of vaccine, medicament or other substance into a patient, the distal end 28 of the needle cap 12 is placed against the patient's skin at the site of the injection. As shown typically in FIG. 3, the distal end 28 of the needle cap 12 includes a substantially flat end wall or surface and a needle aperture in a central portion thereof to allow the needle 16 to pass therethrough. The syringe 32 is then moved toward the patient's skin to move the needle cap 12 from the first position to the second position, as shown in FIGS. 4B-4F, by compressing the needle cap 12 against the bias of its spring 24 and pushing the needle 16 through the distal end of the needle cap and into the patient's skin. Continued movement of the syringe 32 toward the patient's skin causes the needle cap 12 to fully compress against the bias of its spring 24 as the needle 16 is pushed fully through the distal end 28 of the needle cap and into the patient's skin. During movement between the first and second positions, the metal needle continues to be concealed from view because the proximal portion is enclosed within the cap and the distal portion is penetrated into the skin underlying and/or adjacent to the distal end of the cap. As indicated above, because the spring constant of the first spring 24 is less than the spring constant of the second spring 98, the first spring 24 is substantially fully compressed (and thus the needle 16 is fully penetrated) before compression of the second spring 98 is initiated (and thus before the fluid in the compression chamber 100 is dispensed through the dispensing valve 102 and into the needle 16). As shown in FIG. 10A, once the needle cap 12 is fully compressed against the bias of its spring 24 and the needle 16 is fully penetrated through the patient's skin, continued movement of the plunger 94 of the syringe 32 toward the patient's skin moves the plunger against the bias of its spring 98 to fully compress its spring and, in turn, pressurize the dose of fluid in the compression chamber 100 and dispense the dose of fluid from the compression chamber, through the one-way dispensing valve 102, the one-way valve 22 and needle 16 of the device 10, and into the patient. Once the dose is injected, the device 10 and syringe 32 are removed from the patient's skin to thereby allow the needle cap 12 to automatically move under the bias of its spring 98 from the second position to the third position, as shown in FIGS. 4F-4K. As shown in FIG. 4K, in the third position, the needle 16 is received within and enclosed by the needle cap 12, and the needle cap 12 and body 14 are locked in the third position to prevent needle reuse and injury. During movement of the second position to the third position, the cap continues to conceal the needle from view. In addition, any body fluids on the needle or at or about the injection site are enclosed within the cap (with the retracted needle) and otherwise blocked by the device 10 and prevented from contacting the syringe 32. The syringe plunger 94 moves automatically under the bias of its spring 98 from the second or compressed position back to the first or normal position, and during such movement, causes another dose of vaccine, medicament or other substance, to be pulled through the inlet valve 104 and into the compression chamber 100 to ready the syringe to dispense another dose. The used device 10, which is locked in its unusable third position, is inserted through the opening 64 of the sharps container 66 until the protuberances 62, 62 are located within the container, and the syringe 32 is then pulled away from the device 10 to remove the used device from the syringe and dispose of it in the sharps container (FIG. 5D). The device 10 not only conceals the needle from view throughout the injection cycle, but prevents the needle or any body fluids from contaminating the syringe thereby preventing cross contamination from the device or its needle to the syringe, from the device to the healthcare worker, from the patient to the healthcare worker, or from one patient to the next. Yet another advantage is that because the used metal needle is locked in the third position in the enclosure or cap, the needle cannot be reused as encountered with prior art metal needles and syringes.

As can be seen, a significant advantage of the device is that it provides both SIP and needle RUP. Yet another advantage is that the patient never sees the needle because the needle is enclosed within the needle cap whenever the needle is not in use. In the illustrated embodiment, the needle cap is a substantially cylindrical, opaque enclosure that conceals the needle from view; however, as may be recognized by those of ordinary skill in the pertinent art based on the teachings herein, the needle cap may be transparent, translucent, may only partially enclose or block the view of the needle, and otherwise may take any of numerous different configurations that are currently known or that later become known. Yet another advantage of the illustrated embodiment is that the multiple dose syringe is made of only a few parts, including the body 88, the plunger 94 with co-molded bellows 106, elastic valve member 102 and insert molded valve seat 110, a connector 146 (FIG. 8) located at the proximal end of the compression chamber 100 and forming the valve seat of the inlet valve 104, spring 98 and inlet conduit 116. In the illustrated embodiment, the distal end of the inlet conduit 116 is fixedly sealed to the connector 146. This, in combination with the single-use connector 132, prevents reuse of the syringe 32 after dispensing the multiple doses in the respective pouch 130. In other words, once a pouch 130 is emptied, the single-use connector 132 and inlet conduct 116 cannot be removed from the syringe 32 without destruction or damage thereto. Thus, the entire combination or assembly of the pouch 132, single-use connector 132, inlet conduit 116 and syringe 32 is sealed with respect to ambient atmosphere and cannot be reused once the pouch is emptied. Yet another advantage is that there is no sliding seal between the syringe plunger and the body or barrel, as in prior art syringes, and therefore the force required to actuate the plunger and inject a dose is approximately equal to the force required to compress the spring 98, which is substantially constant and thus user independent. A further advantage is that the needle 16 may be a conventional, off the shelf needle, thus reducing manufacturing cost. One such needle is a conventional 20 gauge needle; however, other needle gauges equally may be used, including 23 gauge needles and numerous other needle gauges. As another example, for intramuscular injection, a 23 gauge, 16 millimeter needle may be used.

The multiple dose pouch, including the filling and dispensing ports and penetrable and resealable septum(s) thereof, the devices and methods for making and sterile filling such pouches, and the sterile connector, may take the form of any of numerous different pouches, devices and methods, and sterile connectors, that are currently known, or that later become known, including as set forth in the following patents and patent applications filed in the name of the inventor of this patent application, each of which is hereby expressly incorporated by reference in its entirety as part of the present disclosure: U.S. patent application Ser. No. 16/797,570, filed Feb. 21, 2020, entitled “Pouch with Fitment and Method of Making Same,” which is a continuation of similarly-titled U.S. patent application Ser. No. 15/410,740, filed Jan. 19, 2017, which claims the benefit of U.S. Provisional Patent Application No. 62/295,139, filed Feb. 14, 2016; U.S. Provisional Patent Application No. 62/298,214, filed Feb. 22, 2016 and U.S. Provisional Patent Application No. 62/323,561, filed Apr. 15, 2016, each of which is entitled “Pouch With Over-Molded Fitment And Method Of Making Same”; U.S. Provisional Patent Application No. 62/280,700, filed Jan. 19, 2016, entitled “Pouch with Heat-Sealed External Fitment,” and U.S. Provisional Patent Application No. 62/448,315, filed Jan. 19, 2017, entitled “Pouch With Fitment And Method Of Making Same”; U.S. patent application Ser. No. 14/990,778, filed Jan. 7, 2016, entitled “Pouch with Sealed Fitment and Method,” now U.S. Pat. No. 10,500,132, which claims the benefit of similarly-titled U.S. Provisional Patent Application No. 62/100,725, filed Jan. 7, 2015; U.S. patent application Ser. No. 16/630,940, filed Jan. 14, 2020, entitled “Apparatus and Method for Tracing Primary and Process Devices, and Closed Sterile Transfer Formulation and Filling in Connection with the Traced Devices,” which is a U.S. national stage of international application no. PCT/US2018/042196, filed Jul. 14, 2018, entitled “Apparatus and Method for Tracing Primary and Process Devices, and Closed Sterile Transfer Formulation,” which claims the benefit of U.S. Provisional Patent Application No. 62/534,152, filed Jul. 18, 2017, entitled “Apparatus and Method for Tracing Primary and Process Devices, and Closed Sterile Transfer Formulation and Filling in Connection with the Traced Devices,” and similarly-titled U.S. Provisional Patent Application No. 62/532,972, filed Jul. 14, 2017; U.S. patent application Ser. No. 16/035,627, filed Jul. 14, 2018, entitled “Apparatus and Method for Tracing Primary and Process Devices, and Closed Sterile Transfer Formulation and Filling in Connection with the Traced Devices,” which claims the benefit of similarly-titled U.S. Provisional Patent Application No. 62/534,152, filed Jul. 18, 2017, and similarly-titled U.S. Provisional Patent Application No. 62/532,972, filed Jul. 14, 2017; and U.S. patent application Ser. No. 15/410,762, filed Jan. 19, 2017, entitled “Single Use Connectors,” now U.S. Pat. No. 10,426,701, which claims the benefit of similarly-titled U.S. Provisional Patent Application No. 62/280,693, filed Jan. 19, 2016.

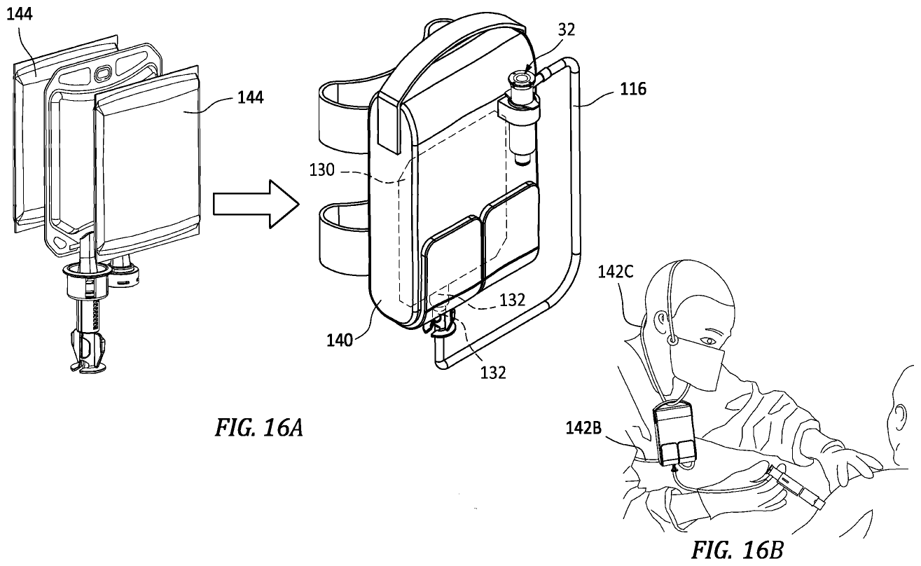

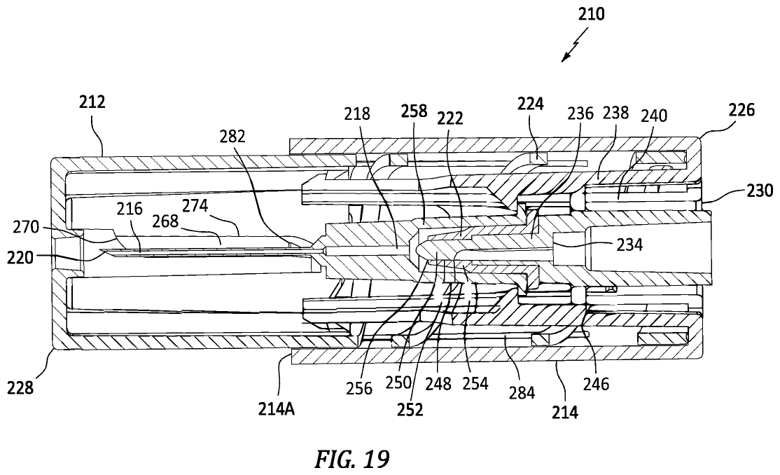

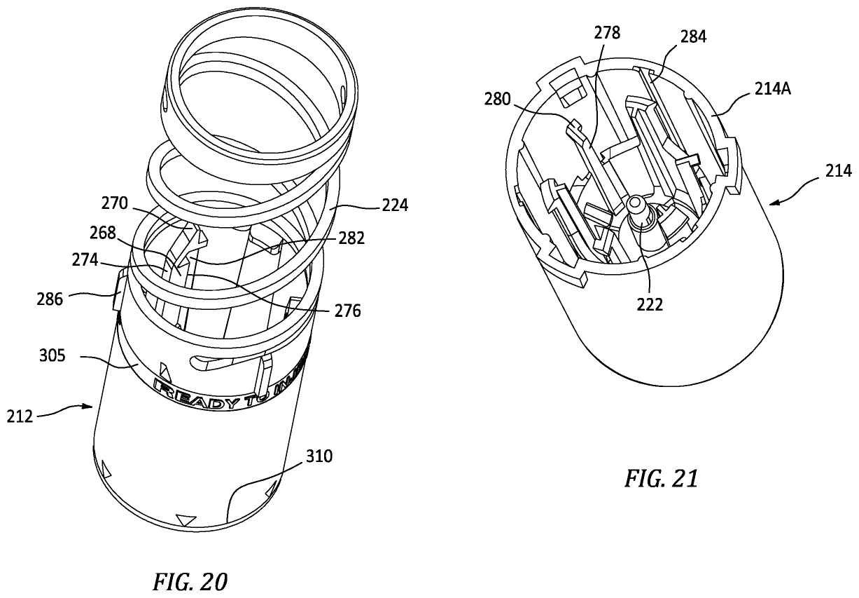

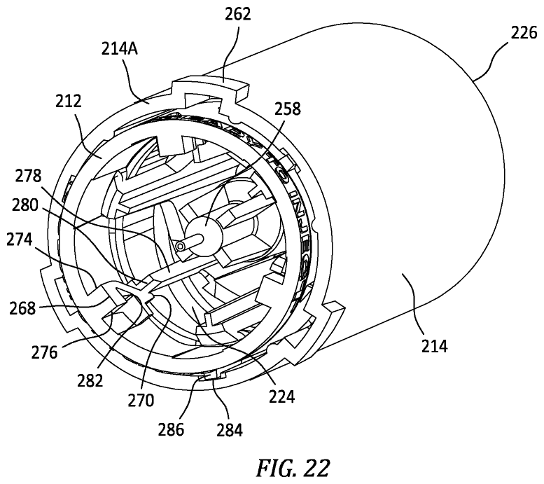

FIGS. 17-22 illustrate another device embodying the invention for preventing needle reuse and injury, indicated generally by the reference numeral 210. Device 210 is similar to device 10 described above, and therefore like reference numerals preceded by the numeral “2” are used to indicate like elements. In the illustrated embodiment, the cap 212 has three locking members 268 circumferentially spaced within the cap 212 at approximately 120 degrees from each other. The body includes three locking arms 278, each locking arm 278 is located so as to correspond to one of the locking members 268. The locking members 268 and locking arms 278 function in a similar manner as do the locking member(s) 68 and locking arm(s) 78 of device 10. The device 210 as seen in, e.g., FIGS. 17, 18 and 19, is in the first position wherein the needle 216 is received within and enclosed by the cap 212. During relative movement of the cap 212 and body 214 from the first position toward the second position in which the needle 216 is passed though or exposed outside of the cap 212, the locking arm 278 slidably contacts the first side 274 of the locking member 268. After the locking arm 278 is located distally relative to the distal end 272 of the locking member 268, and during relative movement of the cap 212 and body 214 from the second position toward the third position wherein the needle 216 is located within and enclosed by the cap 212, the locking arm 278 flexes laterally and slidably contacts the second side 276 of the locking member 268 until the distal end 280 of the locking arm 278 is biased or otherwise moves into the recess 282 of the locking member to thereby lock the cap 212 and body 214 in the third position.

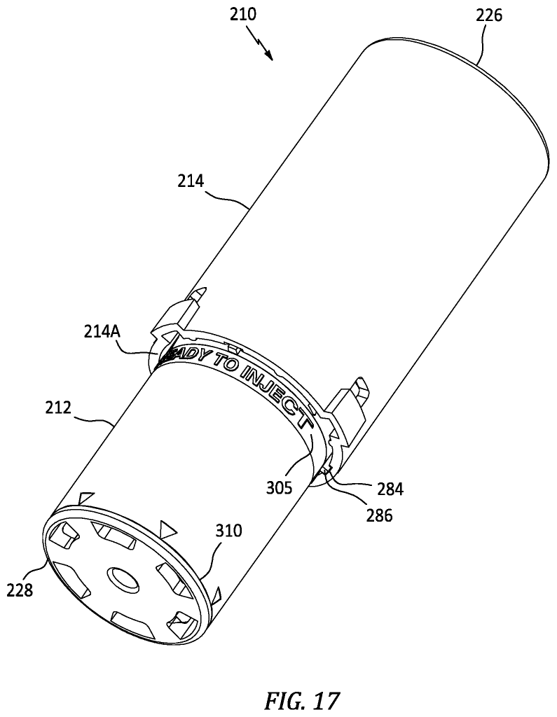

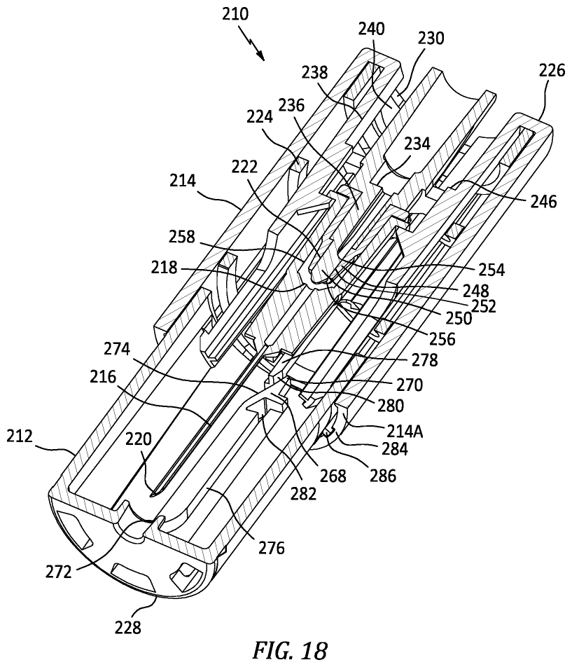

As best seen in FIG. 18, the recess 282 is distally spaced relative to the locking arm 278, and more specifically, relative to its distal end 280, when the cap 212 and body 214 are in the first position. Accordingly, in the locked, third position, the cap 212 is partially retracted into the body 214 as compared to first position. So that the needle 216 is located within and enclosed by the cap 212 in the third position, the cap 212 is provided with a sufficient axial length as compared to the axial length of the needle 216 so that, though the cap 212 does not return to the same position as in the first position, the needle 216 is sufficiently retracted to be located within and enclosed by the cap 212. For example, as can be seen by comparison of FIG. 1 and FIG. 19, the cap 212 of device 210 is longer relative to the needle 216 than the cap 12 is relative to the needle 16 of device 10. This aspect the cap 210 also provides, as compared to cap 10, increased protection against a partial, e.g., accidental, relative movement of the cap 212 and body 214 that will expose the needle 216, which presents a risk of injury to a patient or other user, and/or a risk of contamination of the needle 216.

As best seen in FIG. 17, the exterior of the cap 212 includes a first status indicator 305 and a second status indicator 310, each of which indicates the status of the device 210. In the first position, the first status indicator 305 is visible to a user, indicating that the device 210 is in the first position and may be used to inject a patient. In the illustrated embodiment, the first status indicator constitutes the words “Ready to Inject.” However, the first status indicator 305 may take another form or configuration suitable to indicate the status of the device 210.

During movement from the first position toward the second position, the cap 212 retracts relative to the body 214 such that the second status indicator 310 moves closer to the distal end 214a of the body 214. The second status indicator 310 is located on the cap 212 at a position where, when the second status indicator 310 and the distal end 214a of the body 214 are substantially coincident or aligned, a sufficient or desired length of the needle 216 has passed through the cap 212, e.g., penetrated into the patient, thus indicating that the cap 212 and/or body has moved sufficiently to the second position. In the illustrated embodiment, the second status indicator constitutes a circumferential marking on the cap 212. However, the second status indicator 310 may take another form or configuration suitable to indicate the status of the device 210.

As discussed above, the axial offset of the recess 282 relative to the locking arm 278 (in the first position) results in that, in the third position, the cap 212 is partially retracted into the body 214 as compared to first position. Thus, in the third position, the first status indicator 305 is located within the body 214 and is not visible by a user, indicating that the device 210 is in the locked/third position, or otherwise is not able to be used to inject a patient.

In another aspect, the device 210 has three protuberances (not shown) engageable with the opening 64 in the waste or sharps container 66 to remove the used device 210 from the syringe 32 and dispose of same in the same or similar manner as described above with respect to device 10. The protuberances are circumferentially spaced about the exterior of the body 214 at approximately 120 degrees from each other. However, those of ordinary skill in the art should recognize that device 210 may have more or fewer protuberances than three, and the protuberances may be spaced or arranged on the body 214 in any suitable manner.

In device 210, the one-way valve is received within the base 258 of the needle 216. The one-way valve 212 has a different configuration than valve 22 of device 10. Valve 222 has an elastic valve member 248 engaging the exterior surface of the relatively rigid valve seat 250, thereby forming an axially-elongated, normally-closed valve seam 252 therebetween, with a valve inlet 254 located at a proximal end of the valve seam 252, and a valve outlet 256 located at a distal end of the valve seam 252. The elastic valve member 248 defines an interference fit with the valve seat 250, and defines a valve-opening pressure based on, at least in part, the degree of interference between the elastic valve member 248 and valve seat 250. In the illustrated embodiment, the valve seat 250 is defined by an annular or circumferential surface, and is substantially cylindrical along the length of the valve seam 252. However, one of ordinary skill in the art should understand that the valve seat may have a different shape, e.g., tapered or frustoconical, to provide the valve 222 with desired opening and closing characteristics. Also in the illustrated embodiment, the valve member 248 is tapered or decreases in thickness in a direction from the valve inlet 254 toward the valve outlet 256. In this configuration, the valve opening pressure is greater at the valve inlet 254 than the outlet valve 256. Thus, after fluid is passed through the valve seam 252, the valve seam progressively closes starting from the valve inlet 254 and toward the valve outlet 256, preventing fluid from passing upstream (proximally) through the valve 222, which can contaminate the syringe 32 and/or source of fluid.

As should be recognized by those of ordinary skill in the art, though, the invention is not limited to the one-way valve shown in the figures herein, and may take any of numerous different configurations that are currently known, or that later become known, including, but not limited to, as set forth in the following patents and patent applications, each of which is hereby expressly incorporated by reference in its entirety as part of the present disclosure: U.S. Pat. No. 6,892,906, entitled “Container And Valve Assembly For Storing And Dispensing Substances, And Related Method,” U.S. Pat. No. 7,665,923, entitled “Laterally-Actuated Dispenser with One-Way Valve for Storing and Dispensing Metered Amounts of Substances,” and U.S. Pat. No. 7,845,517, entitled “Container And One-Way Valve Assembly For Storing And Dispensing Substances, And Related Method.”

As may be recognized by those of ordinary skill in the pertinent art based on the teachings herein, numerous changes, improvements, modifications, additions and deletions may be made to the above-described and other embodiments of the present invention without departing from the scope of the invention. For example, the components of the device, syringe, pouch, connector and/or harness, may take any of numerous different configurations and may be made of any of numerous materials that are currently known or later become known, and features may be added to or removed therefrom, without departing the from the scope of the invention. In addition, the device for preventing needle reuse or injury need not be used with a multiple dose syringe, but may be used with a conventional syringe, such as a disposable syringe including a barrel, plunger and device connection on the distal end of the barrel that connects to a device for preventing needle reuse and injury. Accordingly, this detailed description of embodiments is to be taken in an illustrative as opposed to a limiting sense.

What is claimed is:

1. A device for preventing needle reuse and injury, comprising:
a needle enclosure;
a body;
a metal needle mounted between the needle enclosure and body, wherein the metal needle defines an inlet and an outlet, and the needle enclosure and/or the body is movable relative to the other from (i) a first position wherein the metal needle is received within and enclosed by the needle enclosure to prevent needle injury; (ii) to a second position wherein at least a portion of the metal needle passes through the needle enclosure to penetrate tissue underlying or in contact therewith; and (iii) to a third position wherein the metal needle is received or retracted within and enclosed by the needle enclosure, and the needle enclosure and body are locked in the third position to prevent needle reuse and injury; and
a one-way valve in fluid communication with the outlet of the metal needle, wherein the one-way valve allows liquid flow therethrough in a direction from the inlet toward the outlet of the needle, and substantially prevents liquid flow therethrough in a direction from the outlet toward the inlet of the needle, to thereby prevent any liquid within the needle from flowing through the needle inlet and outside the device.

2. A device as defined in claim 1, wherein the one-way valve is an intra-needle valve configured to allow fluid flow therethrough in a direction from the inlet toward the outlet of the needle, and substantially prevent fluid flow therethrough in a direction from the outlet toward the inlet of the needle, to thereby prevent fluid within the needle from flowing through the needle inlet and outside the device.

3. A device as defined in claim 1, further comprising a spring mounted between the needle enclosure and body and biasing the needle enclosure and/or the body in a direction from a proximal end toward a distal end of the device.

4. A device as defined in claim 1, wherein the body includes a syringe mount configured to releasably mount the device to a syringe, an inlet port in fluid communication with the one-way valve to allow fluid to flow from a syringe connected thereto to the one-way valve, a needle support including the needle mounted thereon, and a syringe mounting flange spaced relative to the needle support and defining a gap therebetween for receiving a distal end portion of a syringe and releasably mounting the device thereto.

5. A device as defined in claim 1, wherein the one-way valve includes an elastic valve member, a relatively rigid valve seat, an axially-elongated, normally-closed valve seam therebetween, a valve inlet located at approximately a proximal end of the valve seam, and a valve outlet located at approximately a distal end of the valve seam.

6. A device as defined in claim 5, wherein the elastic valve member defines on a proximal end thereof a recessed surface defining an inlet port in fluid communication with the normally-closed, axially-elongated valve seam.

7. A device as defined in claim 1, wherein the needle enclosure includes a locking member defining a proximal end, a distal end, a first side located between the proximal and distal ends, and a second side opposite the first side and located between the proximal and distal ends, and the body includes a locking arm, wherein the locking arm slidably contacts the first side of the locking member during movement from the first positon to the second position, and slidably contacts the second side of the locking member during movement from the second position to the third position.

8. A device as defined in claim 7, wherein the locking arm is biased into contact with the locking member, the locking arm defines a distal end that slidably contacts the locking member, a recess is defined on one or both of the second side of the locking member and a proximal end thereof, and in the third position, the distal end of the locking arm is biased into the recess to thereby lock the needle enclosure and body in the third position.

9. A device as defined in claim 1, in combination with a syringe including a syringe body defining a distal end and a proximal end, a device mount located on a distal end of the syringe body and releasably engageable with the device body, a plunger received within a distal end of the syringe body, and a seal engageable with the device body to form a substantially fluid-tight seal therebetween.

10. A device and syringe as defined in claim 9, wherein the device further comprises a first spring defining a first spring constant and mounted between the needle enclosure and body and biasing the needle enclosure and/or the body in a direction from a proximal end toward a distal end of the device, and the syringe includes a second spring defining a second spring constant and mounted between the plunger and syringe body, wherein the first spring constant is less than the second spring constant to thereby allow the device to move from the first position to the second position prior to injecting fluid from the syringe through the needle of the device.

11. A device and syringe as defined in claim 9, wherein the syringe defines a compression chamber located between the plunger and a distal end of the syringe body, at least one of the syringe body or plunger is movable relative to the other between a first position wherein the compression chamber defines a first volume and a second position wherein the compression chamber defines a second volume less than the first volume, and the second spring biases the plunger in a direction from the second position toward the first position.

12. A device and syringe as defined in claim 11, wherein the syringe further comprises a one-way dispensing valve in fluid communication with the compression chamber and defining an outlet valve-opening pressure, and a one-way inlet valve in fluid communication with the compression chamber and defining an inlet valve-opening pressure, wherein movement of the syringe body or plunger in a direction from the first position toward the second position pressurizes fluid in the compression chamber to a pressure at or above the outlet valve-opening pressure to flow through the dispensing valve and the device one-way valve and needle, and movement of the syringe body or plunger in a direction from the second position toward the first position opens the syringe inlet valve to allow fluid to flow into the compression chamber and the device one-way valve prevents fluid within the needle from flowing therethrough and into contact with the syringe.

13. A device and syringe as defined in claim 12, wherein the compression chamber is defined by a flexible wall located between the dispensing valve and the inlet valve and defining a variable-volume compression chamber, the plunger is slidably received within the syringe body, the second spring is located between the plunger and syringe body, and the force required to move the plunger or syringe body from the first position to the second position is approximately equal to the force required to displace the spring upon moving between the first and second positions.

14. A device and syringe as defined in claim 12, wherein the one-way dispensing valve includes an elastic valve member, a relatively rigid valve seat, and an axially-elongated, normally-closed valve seam therebetween, and the one-way inlet valve is a flapper valve.

15. A device and syringe as defined in claim 9, in combination with a waste container defining an opening thereto, wherein the body includes on an exterior surface thereof at least one protuberance engageable with the opening to remove the device from the syringe and dispose of same in the waste container.

16. A device and syringe as defined in claim 15, in further combination with a plurality of said devices mounted on a multiple device carrier, wherein each device includes a cover removably attached to a proximal end thereof and configured to maintain the interior of the respective device sterile.

17. A device and syringe as defined in claim 9, in further combination with a multiple dose pouch connected or connectable in fluid communication with the syringe, a container configured to hold the pouch, and one or more straps coupled to the container and configured to support the container and pouch from a person's torso, shoulder, arm, hand, or other body part, or from a stand or table.

18. A device for preventing needle reuse and injury, comprising:
a metal needle defining an inlet and an outlet;
first means for mounting the metal needle thereon and releasably connecting the device to a syringe;
second means movable relative to the first means from (i) a first position for enclosing the metal needle and preventing needle injury; (ii) to a second position for allowing at least a portion of the metal needle to pass through a distal portion of the second means and penetrate tissue underlying or in contact therewith; and (iii) to a third position enclosing the metal needle and locking the first and second means in the third position for preventing needle reuse and injury; and
third means in fluid communication with the outlet of the metal needle for allowing liquid flow therethrough in a direction from the inlet toward the outlet of the needle and substantially preventing liquid flow therethrough in a direction from the outlet toward the inlet of the needle and for preventing any liquid within the needle from flowing through the needle inlet and outside the device.

19. A device as defined in claim 18, wherein the first means a device body, the second means is a needle enclosure, and the third means is a one-way valve.

20. A device as defined in claim 18, further comprising fourth means for biasing the first means and/or the second means in a direction from a proximal end toward a distal end of the device.

21. A device as defined in claim 20, in combination with a syringe including a syringe body defining a distal end and a proximal end, and further including fifth means for receiving the device in an opening in a waste container and for removing the device from a syringe and disposing of same in the waste container; sixth means for releasably engaging the first means and releasably mounting the device thereto; seventh means received within a distal end of the first means for manually engaging and injecting fluid from the syringe and through the third means and metal needle of the device; and eighth means for forming a substantially fluid-tight seal between the first means and the syringe.

22. A method comprising:
(i) placing a distal end portion of a needle enclosure in a first position against a subject's skin wherein a metal needle is received within and enclosed by the needle enclosure to prevent needle injury;
(ii) pushing the needle against the bias of a first spring from the first position to a second position wherein the metal needle extends through a distal end portion of the needle enclosure and penetrates skin or tissue underlying or in contact with the distal end portion of the needle enclosure;
(iii) injecting a dose of substance through a one-way valve in fluid communication with the needle, through the needle and into the skin or tissue;
(iv) withdrawing the needle from the skin and allowing the needle enclosure to move under the bias of the spring from the second position to a third position wherein the metal needle is received within and enclosed by the needle enclosure, and the needle enclosure and body are locked in the third position to prevent needle reuse and injury; and
(v) substantially preventing liquid flow through the one-way valve in a direction from an outlet toward an inlet of the needle and preventing liquid in the needle from flowing through the needle inlet and outside the device.

23. A method as defined in claim 22, wherein the needle enclosure conceals the needle from view in the first and third positions.

24. A method as define in claim 23, further comprising removably attaching the device to a syringe, injecting the dose of substance from the syringe, through the one-way valve and needle, and into the skin or tissue, and after the withdrawing step, removing the device from the syringe and preventing with the one-way valve cross-contamination between the needle and syringe.

25. A method as defined in claim 24, wherein the injecting step includes pushing the syringe against the bias of a second spring to inject the dose of substance, wherein the second spring defines a second spring constant or force required to deflect the second spring that is greater than a spring constant or force required to deflect the first spring.
